(19) United States
(12) Patent Application Publication
(10) Pub. No.: US 2026/0248385 A1
(43) Pub. Date: Aug. 27, 2026

(54) SYSTEMS AND METHODS FOR OPTIMIZED RETINAL IMAGE CAPTURE USING DUAL-STAGE AI-BASED QUALITY ASSESSMENT

(71) Applicant: AI Optics Inc., New York, NY (US)

(72) Inventors: Luke Michael Moretti, New York, NY (US); Shiva Sanketh Mathad, San Jose, CA (US); Sagar Soni, Sunnyvale, CA (US)

(21) Appl. No.: 19/547,447

(22) Filed: Feb. 23, 2026

Related U.S. Application Data
(60) Provisional application No. 63/763,496, filed on Feb. 26, 2025.

(51) Int. Cl. A61B 3/14; G06T 7/00; G06V 10/98

(52) U.S. Cl. CPC A61B 3/14; G06T 7/0012; G06V 10/98; G06T 2207/10024; G06T 2207/10048; G06T 2207/20081; G06T 2207/30041; G06T 2207/30168

(57) ABSTRACT

Disclosed in an integrated retinal imaging system designed to enable high-quality, diagnostic image capture with minimal operator intervention. The system can implement a dual-stage Holistic Quality Assessor (HQA), with HQA-1 performing real-time, holistic quality assessments on infrared or near infrared image frames to detect optimal capture conditions, and HQA-2 verifying the diagnostic adequacy of a captured color image. Both HQA-1 and HQA-2 can leverage machine learning or deep-learning models executed on a local hardware accelerator and are configured to provide low-latency and accurate evaluations. By combining dual-stage quality assessment with real-time capture optimization, the system can enable reliable, diagnostic-quality image capture across various clinical settings and user experience levels.

### CROSS-REFERENCE TO RELATED APPLICATIONS

This application claims priority to U.S. Provisional Patent Application No. 63/763,496, filed on Feb. 26, 2025, which is incorporated by reference in its entirety.

### TECHNICAL FIELD

Disclosed are systems and methods that relate to the field of retinal imaging and diagnostic imaging systems, specifically to AI-driven quality assessment and optimized image capture.

### BACKGROUND

Retinal imaging is a critical diagnostic tool for detecting, monitoring, and managing a range of ocular and systemic diseases, such as diabetic retinopathy, glaucoma, age-related macular degeneration, and hypertensive retinopathy. Capturing high-quality, diagnostically sufficient retinal images is essential for accurate diagnosis and treatment planning. However, achieving such quality in retinal imaging presents significant challenges, particularly in non-mydriatic (non-dilated pupil) and handheld imaging settings, where precise alignment, optimal lighting, and timing are often difficult to achieve.

### SUMMARY

The systems, methods, and devices described herein each have several aspects, no single one of which is solely responsible for its desirable attributes. Without limiting the scope of this disclosure, several non-limiting features will now be discussed briefly.

In some implementations, a retinal camera includes: a housing; an infrared (IR) or near infrared (NIR) light source supported by the housing and configured to provide continuous, non-mydriatic illumination for capturing IR or NIR retinal images without pharmacological pupil dilation; a visible light source supported by the housing and configured to provide flash illumination for capturing color retinal images, where a single perceived flash can result in acquisition of one or more frames (for instance, one or more frames of a video), and the device can generate a composite color retinal image from those frames; at least one image sensor supported by the housing and configured to capture IR or NIR retinal images and color retinal images; a user interface supported by the housing; an on-board hardware accelerator supported by the housing and configured to implement: a first Holistic Quality Assessor (HQA-1) including at least one machine learning model that is optimized for embedded inference and trained on a dataset that includes IR or NIR retinal images, the HQA-1 being configured to assess quality of the IR or NIR retinal images captured by the at least one image sensor by: 1) determining whether at least one property of a first IR or NIR retinal image satisfies a first quality threshold and 2) responsive to determining that the at least one property of the first IR or NIR retinal image satisfies the first quality threshold, provide an indication that a color retinal image should be captured and, in parallel, update (i) a feedback buffer that applies temporal smoothing including low-pass filtering of per-frame HQA-1 quality outputs, for example via an exponential moving average of a per-frame quality score or probability, optionally further including hysteresis and/or debounce logic to reduce indicator flicker near a threshold to drive stable UI indicators and (ii) an automated capture buffer that enforces multi-frame validation prior to initiating image capture. The multi-frame validation can include evaluating a rolling history of per-frame HQA-1 quality outputs to confirm that the first quality threshold is satisfied by more than one IR or NIR retinal image captured at different times (for example, for a configurable count of frames within a rolling time window and/or for a configurable run of consecutive frames). In some implementations, the feedback buffer and the automated capture buffer are implemented using a same rolling history of per-frame HQA-1 quality outputs and the temporal smoothing and the multi-frame validation are two different uses of that same rolling history. The on-board hardware accelerator can implement a second Holistic Quality Assessor (HQA-2) including at least one machine learning model that is optimized for embedded inference and trained on a dataset that includes color retinal images exhibiting multiple types of quality defects, the HQA-2 configured to: 1) perform a multi-factor or holistic quality assessment of the color retinal image by determining whether at least one property of the color retinal image satisfies a second quality threshold and 2) responsive to determining that the at least one property of the color retinal image satisfies the second quality threshold, provide an indication the color retinal image is of diagnostic quality; and a processor supported by the housing and configured to communicate with the on-board hardware accelerator and to control the IR or NIR light source, the visible light source, the at least one image sensor, and the user interface, the processor being further configured to: responsive to receiving the indication that the color retinal image should be captured, automatically cause activation of the visible light source and capture of the color retinal image by the at least one image sensor; and responsive to receiving the indication that the color retinal image is of diagnostic quality, cause a user interface to provide a notification that a color retinal image of diagnostic quality has been captured, wherein the HQA-1 and HQA-2 are optimized to perform all image quality assessments locally by the on-board processing circuitry, including the on-board hardware accelerator without transmitting any of the IR or NIR retinal images or any of the color retinal images to any external computing device for quality assessment thereby enabling low-latency local and sequential assessment of retinal image quality by the HQA-1 and HQA-2 and capture of diagnostic quality color retinal images with reduced or minimal manual intervention by an operator.

In certain cases, timing and synchronization for switching between IR video aiming and visible-light capture, and for coordination the flash are effected by firmware on the processor and/or by dedicated timing logic (e.g., Field-Programmable Gate Array (FPGA), Image Signal Processor (ISP), or Application-Specific Integrated Circuit (ASIC)). In some examples, the same functions are implemented solely in software or firmware on a general purpose processor without dedicated logic. The automated capture control can be realized as a state machine which consumes the dual-buffer outputs, prioritizes manual user input.

In some implementations, a method of operating a retinal camera includes: at a first time, by an on-board hardware accelerator: with a first Holistic Quality Assessor (HQA-1) including at least one machine learning model that is optimized for embedded inference and trained on a dataset that includes IR or NIR retinal images, assessing quality of infrared (IR) or near infrared (NIR) retinal images captured as a result of illuminating a retina with continuous, non-mydriatic illumination, the assessing including: 1) determining whether at least one property of a first IR or NIR retinal image satisfies a first quality threshold and 2) responsive to determining that the at least one property of the first IR or NIR retinal image satisfies the first quality threshold, providing an indication that a color retinal image should be captured and concurrently updating (i) a feedback buffer to drive user interface indicator and (ii) an automated capture buffer that requires a configurable count of consecutive sufficient quality frames; and at a second time, by an on-board processor: responsive to receiving the indication that the color retinal image should be captured, automatically causing illumination of the retina with flash illumination and capture of the color retinal image; and at a third time, by the on-board hardware accelerator: with a second Holistic Quality Assessor (HQA-2) including at least one machine learning model that is optimized for embedded inference and trained on a dataset that includes color retinal images exhibiting multiple types of quality defects: 1) determining whether at least one property of the color retinal image satisfies a second quality threshold and 2) responsive to determining that the at least one property of the color retinal image satisfies the second quality threshold, providing an indication the color retinal image is of diagnostic quality and causing the on-board processor to provide a notification that a color retinal image of diagnostic quality has been captured, wherein the HQA-1 and HQA-2 are optimized to perform all image quality assessments locally by the on-board processing circuitry, including the on-board hardware accelerator without transmitting any of the IR or NIR retinal images or any of the color retinal images to any external computing device for quality assessment thereby enabling low-latency local and sequential assessment of retinal image quality by the HQA-1 and HQA-2 and capture of diagnostic quality color retinal images with reduced or minimal manual intervention by an operator.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various aspects of the disclosure will now be described with regard to certain examples and implementations, which are intended to illustrate but not limit the disclosure.

Throughout the drawings, reference numbers may be re-used to indicate correspondence between referenced elements. The drawings are provided to illustrate example implementations described herein and are not intended to limit the scope of the disclosure.

FIGS. 1A and 1B illustrates example retina cameras.

FIG. 2 illustrates a block diagram of various components of a retina camera.

FIG. 3 illustrates a block diagram of optimized image capture with a retina camera.

FIG. 4 illustrates a process for image capture and dual-stage quality assessment.

FIGS. 5A and 5B illustrate infrared or near infrared and color images of a retina.

### DETAILED DESCRIPTION

### Overview

Disclosed approaches address the shortcomings of current retinal imaging technology by introducing an integrated image quality assessment and capture system that facilitates consistent, high-quality image capture. Disclosed approaches can deliver a seamless workflow that enables precise, optimally timed, and high-quality captures for diagnostic imaging with minimal operator expertise.

In some implementations, a dual-stage, AI-powered retinal imaging system can be configured for real-time acquisition and verification of high-quality, diagnostic retinal images. The system can leverage a dual-stage Holistic Quality Assessment (HQA), powered by deep learning (or machine learning) models executed on, and optimized for, one or more hardware accelerators (such as, one or more graphics processing units (GPUs), Neural processing Units (NPUs), tensor processing units (TPUs)), and managed by one or more controllers to facilitate efficient, accurate, and user-friendly imaging with minimal operator intervention. The dual-stage HQA is configured to facilitate capture of an optimal infrared image—corresponding to a color image of adequate quality—is captured effectively and accurately while confirming that the resulting color image meets diagnostic standards, providing a significant advantage for novice operators and handheld applications.

The retinal imaging system can facilitate high-quality, diagnostic image capture through automated, real-time quality assessment and optimized capture functionality and imaging workflow. The system can incorporate a comprehensive control framework implemented by a controller, which orchestrates each phase of the imaging workflow, including—pre-capture quality assessment, real-time parameter optimization, image capture, and post-capture verification—in an iterative loop. By coordinating each of these functions, the controller can enable an efficient and precise imaging process that minimizes the need for operator intervention and specialized skill. This approach is especially valuable for novice users and dynamic settings, such as handheld applications.

The dual-stage HQA can provide both pre-capture and post-capture quality assessments. The first stage, HQA-1, continuously evaluates infrared (IR) or near infrared (NIR) image frames in real time to determine whether the current imaging conditions are likely to yield a diagnostic-quality color image. In certain implementations, GPU-accelerated deep learning models are employed to perform a holistic assessment that captures complex image characteristics collectively, facilitating a nuanced and accurate evaluation. Other implementations might have deep-learning models accelerated and optimized for use on other dedicated processing hardware, including Field-Programmable Gate Array (FPGA), Tensor Processing Unit (TPU), and Neural Processing Unit (NPU). When optimal conditions are detected, HQA-1 can indicate to the controller to initiate color image capture at the ideal moment, configured to yield diagnostic-quality results.

In some implementations, the system employs a buffering architecture that runs in parallel with HQA-1 to both stabilize operator feedback and to govern automatic capture: a first feedback buffer ingests the per-frame quality outputs from HQA-1 and applies temporal smoothing including low-pass filtering, for example by applying an exponential moving average to a per-frame HQA-1 quality score or probability, optionally with hysteresis and/or debounce logic so that a user interface indicator remains stable when the quality output hovers near a threshold, so that the feedback indicator remains steady and intuitive despite moment-to-moment fluctuations in the live IR or NIR video. For example, the feedback indicator may comprise a dot displayed on the user interface, the dot being red when HQA-1 outputs insufficient quality and the dot being green when HQA-1 outputs sufficient quality. Concurrently, a second automated capture buffer is used to enforce multi-frame consistency before initiating capture based on the rolling history (for example, requiring that sufficient quality be achieved for a configurable count of frames within a rolling time window and/or for a configurable run of consecutive frames) before arming and issuing a capture command, immediately resetting on any insufficient quality frame to avoid false triggers. The first buffer smooths operator feedback; the second buffer enforces a multi-frame validation policy before arming capture. In some implementations, the first feedback buffer and the second automated capture buffer may be implemented using a same rolling history buffer (or a same data structure storing the rolling history of HQA-1 outputs), with temporal smoothing used to drive the operator-facing indicator and multi-frame validation used to gate capture. In certain implementations, the second buffer may further incorporate motion stability gating e.g., optical-flow based eye/hand motion estimation or fast image registration between successive IR or NIR frames configured to verify that the consecutive quality streak occurs during subject/device stability, thereby improving the likelihood that the subsequently captured color image (single-frame or composite from multiple exposures perceived as one flash) is diagnostically usable. The architecture can decouple perceptual feedback from automation logic allowing each use of the rolling history to be tuned for its respective purpose of responsiveness vs. reliability and can be realized in software on the on-board hardware accelerator with optional timing and synchronization logic.

In some cases, a capture gate is implemented by a controller and consumes HQA-1 quality outputs and motion stability signals and applies multi-frame validation (such as, a simple state machine or conditional logic) prior to permitting visible-light capture. In some examples, the capture gate is implemented with dedicated timing logic, such as an ISP, FPGA, or ASIC, that is configured to evaluate buffered HQA-1 results and stability metrics with deterministic timing aligned to sensor exposure cycles. Alternatively, in some implementations, HQA-1 internally evaluates multiple IR and NIR frames and enforces a multi-frame validation policy itself, and in such cases, a separate capture gate is not used.

In certain implementations, the capture gate is not limited to a strict consecutive-frame validation policy. For example, the capture gate may require that the first quality threshold be satisfied for at least two IR or NIR frames captured at different times within a rolling time window, and the frames need not be consecutive. In further implementations, the capture gate may require satisfaction for a configurable count of frames within a time window, a majority-vote or percentile criterion over a window of frames, a running average or hysteresis applied to a quality score, or other multi-frame validation logic that reduces false triggers caused by transient single-frame artifacts. The capture gate may be implemented as a state machine that consumes HQA-1 outputs (and optionally motion stability signals) to produce an “armed” state that permits visible-light capture. In cases where HQA-1 itself performs multi-frame evaluation and gating of capture conditions, the separate capture gate module is omitted, and the controller proceeds upon receiving the HQA-1 “armed” indication.

In certain implementations, motion stability verification can incorporate inertial sensing from one or more on-board motion sensors, such as an accelerometer and/or gyroscope. For example, the automated capture buffer may require that a measured angular velocity and/or linear acceleration magnitude remain below a stability threshold for a configurable time window that overlaps the consecutive-frame quality requirement. In some implementations, a composite stability score is computed by fusing or combining (i) optical-flow derived motion estimates from the IR or NIR video stream (or other image-to-image motion estimates, including fast registration between successive frames) and (ii) inertial measurements from the one or more motion sensors to estimate device motion (such as, angular velocity or linear acceleration). For example, fusion can produce a composite stability score. As another example, fusion can be implemented by applying threshold logic in which each of the image-derived motion estimate and the inertial measurement(s) are required to satisfy corresponding limits (such as, be below a corresponding threshold). Optical-flow derived motion estimates or other image-to-image motion estimates can estimate how much the eye or device appears to move. This can yield a motion magnitude/trajectory derived directly from the video stream and can indirectly capture blur/motion artifacts). Automated capture can be permitted when the composite stability score satisfies a stability criterion or threshold. This combination of complementary motion estimates can reduce false triggers caused by transient scene motion, operator tremor, or subject movement. In summary, motion stability verification may not allow HQA-1 to initiate color image capture unless motion satisfies a threshold (such as, is below a threshold, where motion can be inferred either from frame-to-frame image motion and/or directly from the one or more motion sensors.

Following image capture, the second stage, HQA-2, can perform a comprehensive quality verification on the captured color image, providing immediate feedback on its diagnostic adequacy. HQA-2’s output can be displayed to the operator on a user interface, offering actionable guidance on whether to retain or retake the image. This feedback can reduce the likelihood of missed diagnoses due to inadequate image quality and minimizes the need for rigorous manual assessments. In certain implementations, HQA-2’s output also enables continuous dataset improvement by labeling corresponding IR or NIR frames, allowing further refinement of HQA-1’s model over time.

In some implementations, a Real-Time Imaging Optimizer (RIO) can be implemented to perform dynamic adjustments to imaging parameters (such as, focus or illumination) based on real-time feedback from HQA-1. RIO can operate selectively and can be integrated with the GPU for ultra-low latency, configured to keep imaging conditions optimized at the moment of capture. This real-time parameter adjustment can be particularly valuable in handheld applications, where conditions can shift rapidly, and low-latency response is critical.

By combining dual-stage quality assessment, real-time capture optimization, and automated feedback, disclosed approaches can create a reliable, responsive, and efficient imaging process that consistently delivers diagnostic-quality retinal images. The integrated control framework can enable high-quality imaging across diverse clinical settings and user experience levels, representing a significant advancement in retinal imaging technology.

The dual-stage quality assessment process—comprising HQA-1’s real-time evaluation and HQA-2’s post-capture verification—provides unique advantages over existing systems. HQA-1’s holistic assessment of IR or NIR frames can facilitate capture of the optimal color image (single or composite from multiple frames) without relying on precise timing from the operator, while HQA-2 is configured to verify that each captured color image meets diagnostic standards. This dual-stage, AI-driven assessment provides the high level of quality control needed to support novice operators in producing high-quality images consistently.

Additionally, the disclosed quality assessment approaches surpass the simplistic parameter-specific checks commonly used in existing systems. By evaluating complex image characteristics holistically, disclosed approaches provide a level of nuanced quality assessment that is both accurate and robust, mirroring the skill and judgment of an experienced clinician. This integrated, sophisticated approach greatly reduces the need for retakes, enhances workflow efficiency, and minimizes reliance on operator skill, thereby delivering a significant advancement over existing retinal imaging systems.

With its dual-stage quality assessment, real-time parameter optimization, and automated capture initiation, disclosed approaches provide a reliable, comprehensive solution for capturing high-quality retinal images in both handheld and tabletop applications.

Challenges with implementing the dual-stage HQA on a portable retina camera include, among others, memory constraints, power demand, latency, and thermal management.

Large AI models with extensive parameters or high-resolution video feeds may exhaust available on-board memory, causing slowdowns or inference failures. Also, AI inference typically requires high memory bandwidth for transferring input data to a processor (such as, a GPU), storing intermediate computations, and writing back results. Video streams, especially high-resolution feeds, further stress memory bandwidth. Repeated allocation and de-allocation of memory for input data, intermediate tensors, and outputs can also lead to fragmentation, reducing available contiguous memory, making it harder to allocate memory for large tensors required by AI models.

Solutions to memory constraints can include one or more of model optimization, implementing memory pools to pre-allocate large memory buffers to handle frequently used tensors and video data, reusing memory allocated for intermediate tensors across different inference cycles when possible by overwriting tensor buffers once they are no longer needed, utilizing a unified CPU/GPU memory to reduce duplication and overhead for copying data between CPU and GPU, or using pinned memory for input data transfer to the GPU, which avoids paging and is configured to provide faster data transfers.

Real-time AI inference especially on live video feeds, may tend to demand continuous GPU/CPU usage, significantly increasing power draw. Sustained high loads stress the battery and power regulator, risking voltage drops, system resets, and reduced battery lifespan. Also, sudden workload changes—such as, switching models or inference tasks—can create transient peaks beyond the system’s power handling capacity. This can cause power supply instability and performance throttling.

Power management can be optimized with regulators configured to provide stable power delivery across varying battery voltages, additional capacitors near the GPU to buffer transients and maintain voltage stability and optimizing energy-efficient GPU acceleration. Dynamic adjustment of power modes based on workload, as well as reducing GPU/CPU usage during idle or light tasks to conserve energy can be implemented through firmware and software controls. In some cases, a secondary power source (such as, a second battery) may be added in parallel with the primary power source to increase capacity and reduce stress on the primary system.

Real-time processing may require rapid data movement between one or more sensors, memory, and AI accelerators. This can cause insufficient memory bandwidth or bottlenecks in communication buses can increase latency. Additionally, large/complex AI models with high parameter counts may require significant computational resources, leading to delays in inference.

To address these problems, direct memory access (DMA) can be used for data transfer between one or more sensors, memory, and GPU, thus bypassing the CPU and reducing latency. In some implementations, zero-copy or unified memory pipelines are employed to further reduce data movement and latency.

Continuous GPU/CPU usage may generate significant heat, risking thermal throttling and performance degradation. Active cooling methods, such as fans or solid-state cooling, along with electronic board designs that incorporate thermal vias and copper or aluminum heat dissipators near high-power components, can effectively mitigate these thermal issues.

### Medical Diagnostics Devices with On-board AI Capabilities

A device with integrated artificial intelligence (AI) can be used to assess a patient’s body part to detect a disease. The device can be portable or handheld by a user (which can be a patient or a healthcare provider). For example, the device can be a retina camera (or retinal camera) configured to assess a patient’s eye (or retina) and, by using an on-board AI retinal disease detection system, provide real-time analysis and diagnosis of disease that caused changes to the patient’s retina. Easy and comfortable visualization of the patient’s retina can be facilitated using such retina camera, which can be placed over the patient’s eye, display the retina image on a high-resolution display, potentially with screenshot capabilities, analyze a captured image by the on-board AI system, and provide determination of presence of a disease.

Such retina camera can perform data collection, processing, and diagnostics tasks on-board without the need to connect to another computing device or to cloud computing services. This approach can avoid potential interruptions of the clinical workflow when using cloud-based solutions, which involve transfer of data over the network and, accordingly, rely on network connectivity. This approach can facilitate faster processing because the device can continually acquire and process images without needing intermediary upload/download steps, which can be slow. Such retina camera can potentially improve accuracy (for instance, as compared to retina cameras that rely on a human to perform analysis), facilitate usability (for example, because no connectivity is used to transfer data for analysis or transfer results of the analysis), provide diagnostic results in real-time, facilitate security and guard patient privacy (for example, because data is not transferred to another computing device), or the like. Such retina camera can be used in many settings, including places where network connectivity is unreliable or lacking.

Such retina camera can allow for better data capture and analysis, facilitate improvement of diagnostic sensitivity and specificity, and improve disease diagnosis in patients. Existing fundus cameras can lack one or more of portability, display, on-board AI capabilities, etc. or require one or more of network connectivity for sharing data, another device (such as, mobile phone or computing device) to view collected data, rigorous training of the user, etc. In contrast, allowing for high-quality retinal viewing and image capturing with faster analysis and detection of the presence of disease via on-board AI system and image-sharing capabilities, the retina cameras described herein can potentially provide improved functionality, utility, and security. Such retina camera can be used in hospitals, clinics, and/or at home. The retina cameras or other instruments described herein, however, need not include each of the features and advantages recited herein but can possibly include any individual one of these features and advantages or can alternatively include any combination thereof.

As another example, the device can be an otoscope configured to assess a patient’s ear and, by using an on-board artificial intelligence (AI) ear disease detection system, possibly provide immediate analysis and/or diagnosis of diseases of the patient’s ear. Such an otoscope can have one or more advantages described above or elsewhere in this disclosure. As yet another example, the device can be a dermatology scope configured to assess a patient’s skin and, by using an on-board artificial intelligence (AI) skin disease detection system, possibly provide immediate analysis and/or diagnosis of diseases of the patient’s skin. Such a dermatology scope can have one or more advantages described above or elsewhere in this disclosure.

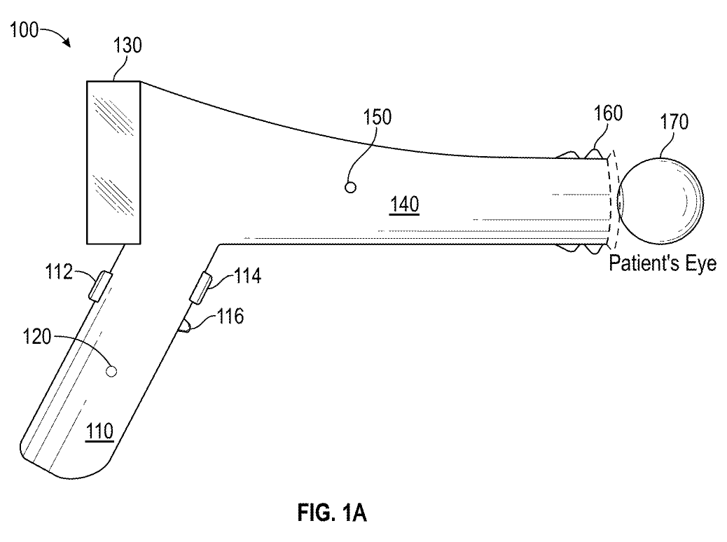

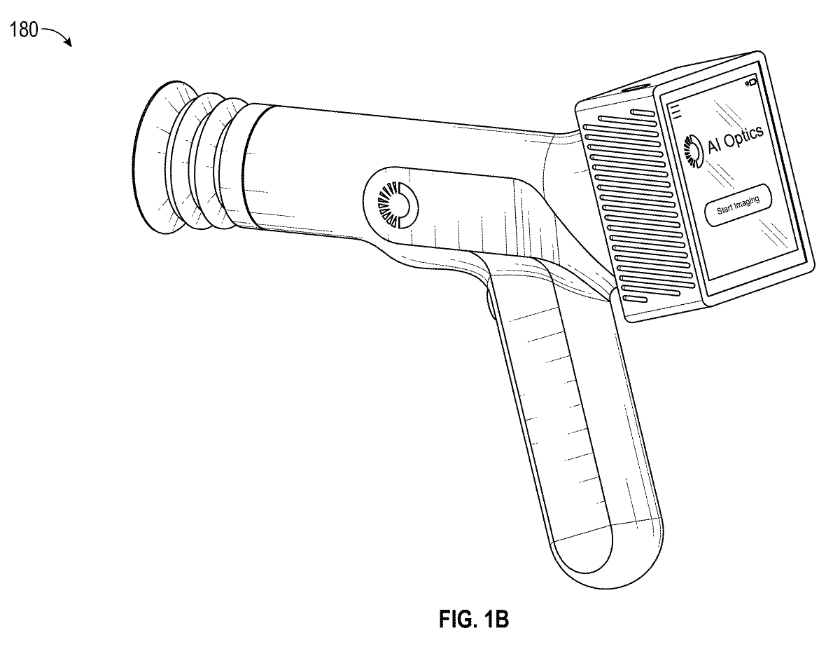

FIG. 1A illustrates an example retina camera 100. A housing of the retina camera 100 can include a handle 110 and a body 140 (in some cases, the body can be barrel-shaped). The handle 110 can optionally support one or more of power source, imaging optics, or electronics 120. The handle 110 can also possibly support one or more user inputs, such as a toggle control 112, a camera control 114, an optics control 116, or the like. Toggle control 112 can be used to facilitate operating a display 130 in case of a malfunction. For example, toggle control 112 can facilitate manual scrolling of the display, switching between portrait or landscape mode, or the like. Toggle control 112 can be a button. Toggle control 112 can be positioned to be accessible by a user’s thumb. Camera control 114 can facilitate capturing video or an image.

In some cases, actuation of the camera control 114 generates a capture request (or capture trigger) that is latched by a controller (for example, an ICS controller as described herein) such that the capture request remains asserted after the initial actuation and independent of continued user pressure on the camera control 114. While the capture request is latched, the system continues IR or NIR acquisition and HQA-1 evaluation and applies multi-frame validation and, optionally motion-stability gating. Visible-light capture is initiated automatically when the capture gate (or HQA-1, in implementations that perform internal gating) indicates capture conditions are satisfied without requiring the operator to re-actuate the camera control 114. The latched capture request can be cleared upon a defined termination condition, such as successful acquisition of a color retinal image that meets diagnostic quality (per HQA-2), a user-initiated cancellation (e.g., by a second actuation or a user-interface command), or expiration of a timeout interval.

In certain implementations, IR or NIR guidance imaging may be initiated responsive to initiation of a screening session (for example, in response to an operator selecting a “Start Screening” control on the display), and the image sensor may continuously capture or generate IR or NIR retinal frames that are streamed to the user interface (such as, shown on the display) and, in parallel, provided to HQA-1 in real time as each of the frames arrives.

In some cases, a separate capture request through a user interface (such as, through the display or the camera control 114) may be used to control at least one aspect of the pipeline for power management and/or workflow control, including gating execution of HQA-1 while IR or NIR capture may continue for display. As another example, the capture request can gate authorization of visible-light flash capture while HQA-1 continues to provide quality feedback on the user interface (such as, on the display). This approach can provide guidance to the user (such as, on-screen guidance) without automatically initiating color image capture. In such implementations, the capture gate, when implemented as a distinct module, can be executed by the controller and consumes HQA-1 outputs.

Camera control 114 can be a button. Camera control 114 can be positioned to be accessible by a user’s index finger (such as, to simulate action of pulling a trigger) or middle finger. Optics control 116 can facilitate adjusting one or more properties of imaging optics, such as illumination adjustment, aperture adjustment, focus adjustment, zoom, etc. Optics control 116 can be a button or a scroll wheel. For example, optics control 116 can focus the imaging optics. Optics control 116 can be positioned to be accessible by a user’s middle finger or index finger.

The retina camera 100 can include the display 130, which can be a liquid crystal display (LCD) or other type of display. The display 130 can be supported by the housing as illustrated in FIG. 1A. For example, the display 130 can be positioned at a proximal end of the body 140. The display 130 can be one or more of a color display, high resolution display, or touch screen display. The display 130 can reproduce one or more images of the patient’s eye 170. The display 130 can allow the user to control one or more image parameters, such as zoom, focus, or the like. The display 130 (which can be a touch screen display) can allow the user to mark whether a captured image is of sufficient quality, select a region of interest, zoom in on the image, or the like. Any of the display or buttons (such as, controls, scroll wheels, or the like) can be individually or collectively referred to as user interface. The body 140 can support one or more of the power source, imaging optics, imaging sensor (or image sensor), electronics 150 or any combination thereof.

A cup 160 can be positioned on (such as, removably attached to) a distal end of the body 140. The cup 160 can be made at least partially from soft and/or elastic material for contacting patient’s eye orbit to facilitate examination of patient’s eye 170. For example, the cup can be made of plastic, rubber, rubber-like, or foam material. Accordingly, the cup 160 can be compressible. The cup 160 can also be disposable or reusable. In some cases, the cup 160 can be sterile. The cup 160 can facilitate one or more of patient comfort, proper device placement, blocking ambient light, or the like. Some designs of the cup can also assist in establishing proper viewing distance for examination of the eye and/or pivoting for panning around the retina.

FIG. 1B illustrates a retina camera 180, which can be similar to the retina camera 100. The retina camera 180 may not include the toggle control 112 or the optics control 116.

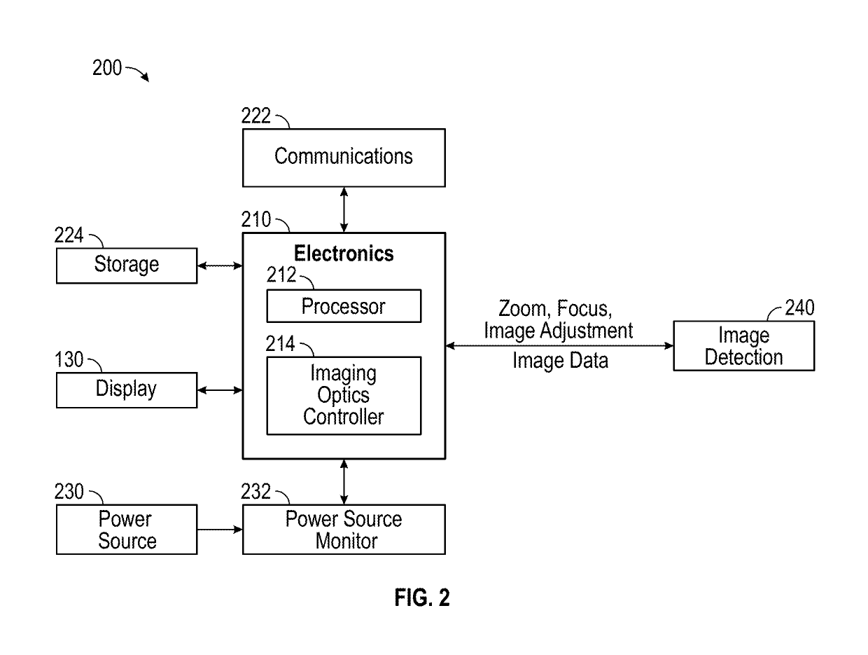

FIG. 2 illustrates a block diagram 200 of various components of the retina camera 100. Power source 230 can be configured to supply power to electronic components of the retina camera 100. Power source 230 can be supported by the handle 110, such as positioned within or attached to the handle 110 or be placed in another position on the retina camera 100. Power source 230 can include one or more batteries (which can be rechargeable). Power source 230 can receive power from a power supply (such as, a USB power supply, AC to DC power converter, or the like). Power source monitor 232 can monitor level of power (such as, one or more of voltage or current) supplied by the power source 230. Power source monitor 232 can be configured to provide one or more indications relating to the state of the power source 230, such as full capacity, low capacity, critical capacity, or the like. One or more indications (or any indications disclosed herein) can be visual, audible, tactile, or the like. Power source monitor 232 can provide one or more indications to electronics 210.

Electronics 210 can be configured to control operation of the retina camera 100. Electronics 210 can include one or more hardware circuit components (such as, one or more controllers or processors 212), which can be positioned on one or more substrates (such as, on a printed circuit board). Electronics 210 can include one or more of at least one graphics processing unit (GPU) or at least one central processing unit (CPU). In certain cases, electronics 210 can also include field-programmable gate arrays (FPGA) or other specialized accelerators to synchronize high speed illumination changes and capture operations, such as switching from IR guidance mode to visible-light flash capture with minimal latency. Electronics 210 can be configured to operate the display 130. Storage 224 can include memory for storing data, such as image data obtained from the patient’s eye 170, one or more parameters of AI detection, or the like. Any suitable type of memory can be used, including volatile or non-volatile memory, such as RAM, ROM, magnetic memory, solid-state memory, magnetoresistive random-access memory (MRAM), or the like. Electronics 210 can be configured to store and retrieve data from the storage 224.

Communications system 222 can be configured to facilitate exchange of data with another computing device (which can be local or remote). Communications system 222 can include one or more of antenna, receiver, or transmitter. In some cases, communications system 222 can support one or more wireless communications protocols, such as WiFi, Bluetooth, NFC, cellular, or the like. In some instances, the communications system can support one or more wired communications protocols, such as USB. Electronics 210 can be configured to operate communications system 222. Electronics 210 can support one or more communications protocols (such as, USB) for exchanging data with another computing device.

Electronics 210 can control one or more imaging detectors 240, which can be configured to facilitate capturing of (or capture) image data of the patient’s eye 170. The imaging detectors 240 can include one or more image sensors and one or more light sources (such as, one or more IR or NIR light sources and one or more visible light sources). Electronics 210 can control one or more parameters of the imaging detectors 240 (for example, zoom, focus, aperture selection, image capture, provide image processing, or the like). Such control can adjust one or more properties of the image of the patient’s eye 170. Electronics 210 can include an imaging optics controller 214 configured to control one or parameters of the imaging detectors 240. Imaging optics controller 214 can control, for example, one or more motor drivers of the imaging detectors 240 to drive motors (for example, to select an aperture, to select lenses that providing zoom, to move of one or more lenses to provide autofocus, to move a detector array or image sensor to provide manual focus or autofocus, or the like). Control of one or more parameters of the imaging detectors 240 can be provided by one or more of user inputs (such as a toggle control 112, a camera control 114, an optics control 116, or the like), display 130, etc. Imaging detectors 240 can provide image data (which can include one or more IR or NIR and color images) to electronics 210. As disclosed herein, electronics 210 can be supported by the retina camera 100. Electronics 210 can be configured to be attached to (such as, connected to) another computing device (such as, mobile phone or server) to perform determination of presence of a disease.

### Challenges of Non-Mydriatic Retinal Imaging

Non-mydriatic retinal imaging systems allow for retinal imaging without requiring the use of pupil-dilating drops, improving patient comfort and workflow efficiency. However, imaging through an undilated pupil reduces the amount of light entering the eye, increasing the likelihood of artifacts such as glare, reflections, incomplete retinal coverage, and misalignment. These limitations can result in images that are diagnostically insufficient and may necessitate retakes, increasing time and effort required for each capture.

These challenges can be especially problematic for novice operators, who may lack the skill to properly align the camera and capture high-quality images within the narrow timing window needed for diagnostic accuracy. In handheld imaging, even slight patient or operator movements can cause the image to go out of focus or misalign, making it difficult to capture an image of sufficient quality. The limited field of view through an undilated pupil further increases the need for precise centering and stability, as even minor shifts can exclude key diagnostic regions. Consequently, capturing a diagnostic-quality photo often requires significant skill and experience, making consistent quality imaging challenging for novice users.

Existing auto-capture systems and methods in retinal imaging are widely considered inadequate due to their reliance on rigid sequences or simplistic assessments of basic imaging characteristics, neither of which provides a comprehensive, accurate assessment of diagnostic image quality.

Many existing auto-capture systems use a rigid sequence, where image capture is triggered upon the completion of a specific step, such as an autofocus protocol. However, simply achieving focus in an IR or NIR frame does not guarantee a high-quality color retinal image, as focus alone is only one element of image quality. This fixed sequence fails to consider other critical quality indicators holistically, such as alignment, glare, and coverage of the retina. As a result, capturing an in-focus IR or NIR frame may not yield a diagnostically sufficient color image, leading to missed opportunities and frequent retakes.

Some existing auto-capture systems may assess frames for one or more basic imaging characteristics (such as, focus, brightness, or contrast) before triggering capture. However, this simplistic approach is limited in accuracy, as it does not account for the complexity of diagnostic image quality in retinal imaging. By focusing on isolated, fixed characteristics, these systems do not conduct a holistic assessment, often leading to color images that lack diagnostic quality. Additionally, the time taken to assess individual characteristics can cause delays, increasing the likelihood of missing optimal alignment as the patient or operator adjusts.

These limitations result in unreliable auto-capture performance, making it difficult for operators—especially novices—to consistently capture high-quality, diagnostically useful images. Even when capture is successful, the absence of integrated per-capture and post capture quality assessment can lead to a higher incidence of false positives or false negatives. Due to these performance issues, many systems either lack auto-capture functionality altogether or operators avoid using it and rely on manual capture despite its challenges.

Basic post-capture quality verification systems and methods also exist, offering a limited check of image adequacy. However, these methods generally rely on simple metrics or subjective operator judgment rather than advanced quality verification. Furthermore, they typically lack the real-time responsiveness needed to provide the operator with immediate, actionable feedback on whether to retain or retake an image. Without an advanced, cohesive quality assessment approach, existing systems fail to provide the comprehensive quality control that today’s diagnostic standards demand.

### Optimized Retinal Imaging

An effective solution to these challenges, in some cases, can combine a real-time, holistic quality assessment capable of analyzing the entire imaging field and determining whether an IR or NIR frame is likely to yield a diagnostically sufficient color image. Such a solution may require a level of comprehensive quality evaluation akin to that of a skilled clinician, assessing multidimensional characteristics collectively, rather than in isolation. This can include simultaneous evaluation of focus, alignment, glare, illumination uniformity, and motion stability, weighted appropriately for their combined influence on the probability of obtaining a diagnostically usable image. An AI-based approach (which can be a deep learning-based approach or a machine learning-based approach) could enable this holistic assessment, providing nuanced evaluation and reducing reliance on simple, sequential quality checks.

This real-time quality assessment may require substantial processing power and ultra-low latency to capture the precise moment when alignment and image conditions are ideal. While the demand for low-latency operation can be important in any setting, it can be particularly crucial in handheld applications, where alignment can shift quickly and unpredictably. Current systems lack the responsiveness and real-time processing capabilities needed to execute this level of quality assessment effectively, limiting their ability to assist the operator at the critical moment.

In some instances, a comprehensive, integrated control framework executed by a controller (which can be referred to as Intelligent Capture System controller) can seamlessly manage each phase of the imaging process—from IR frame quality assessment through real-time parameter optimization to color image quality assessment and verification. The controller can coordinate all system components to work in unison, eliminating or reducing the need for operator intervention and enabling consistently timed, high-quality image capture that meets diagnostic standards. Optimized imaging approaches described herein can, in some cases, be referred to as Intelligent Capture System (ICS) architecture or ICS.

As described herein, the ICS architecture can integrate hardware and firmware/software components including the camera housing, light sources, image sensor, hardware accelerator (such as, embedded GPU-based processing electronics), ICS controller, Real-Time Imaging Optimizer (RIO) for optional parameter adjustments, and the capture functionality. In some configurations, optional dedicated logic (e.g., FPGA or ISP) may be used to coordinate sub-millisecond synchronization between IR or NIR and visible light capture, though the ICS can also achieve this entirely in software or general-purpose processing hardware. This configuration offers flexible implementation options, covering a range of hardware and firmware/software arrangements while configured to provide consistent, high-quality imaging.

HQA-1, which can function as the initial quality assessment stage, can continuously evaluate IR or NIR frames (which may be captured continuously as a video stream) in real-time to assess their potential for producing a diagnostic-quality color image. Upon confirming optimal conditions, HQA-1 can indicate to the ICS controller to activate the capture sequence, which includes visible light illumination and color image acquisition. In some cases, HQA-1’s assessment is gated not only by image quality but also by motion stability metrics derived from optical flow or image registration, configured to permit capture during moments of relative stillness. Following this capture, HQA-2 can perform a secondary quality verification to confirm that the color image meets diagnostic criteria. The ICS controller can coordinate these stages, along with any required parameter adjustments, user feedback, and optional re-initiation of the imaging cycle. ICS with its dual HQA stages configured to capture diagnostic-quality imaging by novice users without requiring advanced skills or extensive training.

### Components of Optimized Retinal Imaging System

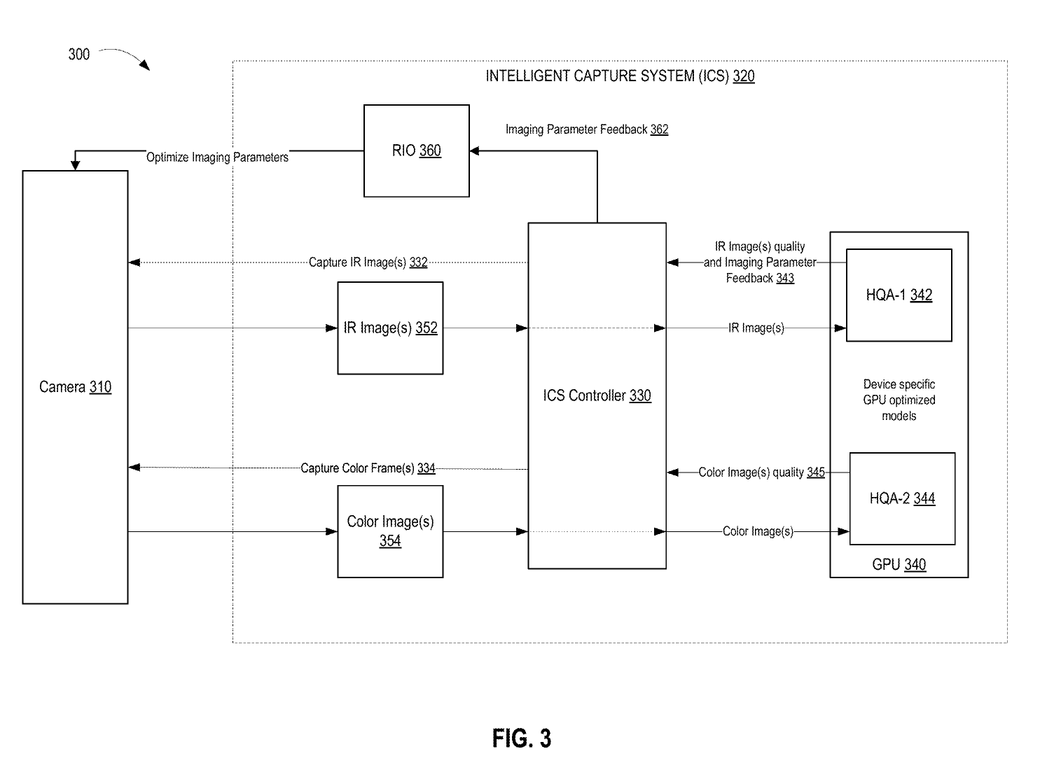

FIG. 3 illustrates a block diagram of a retina camera 300 with ICS 320 illustrating hardware and firmware/software components and data shown as boxes and data paths optimized for low latency shown as arrows.

The retina camera can include a housing, as shown in FIGS. 1A-1B. The housing can be an integrated unit designed for a retinal imaging system, configured to contain and support all components, including light sources, image sensor, processing electronics (including the controller and GPU)), a user interface, a control interface, and a power management system. The housing may be configured for either handheld or tabletop use, depending on user needs and clinical application requirements.

The power management system may be configured to support battery operation for mobile use or a wired setup for continuous operation in fixed settings. In certain implementations, the camera housing can support a user interface that provides real-time feedback and quality indicators, such as output from HQA-2, allowing the operator to assess image quality and sufficiency immediately. This user interface may be a touchscreen, LCD, or another suitable display type, either integrated directly into the housing or provided as a detachable control module. Examples of user interfaces (such as, the display 130) are illustrated in FIGS. 1A-1B.

The retina camera 300 can include one or more light sources (which can be part of a camera module 310 or camera) configured to provide both IR or NIR and visible light (such as, white light), each serving specific roles in facilitating optimal, non-mydriatic retinal imaging. The light sources may be LED-based, laser-based, or employ other suitable technologies, depending on the imaging requirements. In certain cases, the system may use multiple IR or NIR sources and multiple visible light sources, or a single source capable of emitting both IR or NIR and visible light spectrums. The one or more IR or NIR light sources can provide continuous, non-invasive illumination, enabling retinal imaging without the need for pharmacological pupil dilation. This continuous IR or NIR illumination can support the continuous capture and real-time assessment of IR or NIR frames, which are processed by HQA-1 to evaluate their potential for producing a diagnostic-quality color image, which can be subsequently obtained without the need for pharmacological pupil dilation as described herein.

In some implementations, the one or more light sources can produce IR light having a wavelength between about 780nm and about 1mm, NIR light having a wavelength in the range of about 780nm to about 2500nm, and color light having a wavelength in the range of about 380nm to about 780nm.

The one or more visible light sources can be activated when the ICS is ready to capture a high-resolution color image. Upon confirmation from HQA-1 that optimal imaging conditions are met, the one or more visible light sources can emit a brief flash to capture a diagnostic image. In certain cases, this flash may be accompanied by rapid acquisition of multiple closely spaced frames, which are then combined on-device into a composite image that reduces motion blur, improves contrast and reduces artifacts across retinal regions. This flash can be calibrated for brightness, duration, and spectral quality and is configured to provide high diagnostic clarity while maintaining patient comfort. The calibration parameters of the one or more visible light sources may be adjustable to suit various diagnostic needs, enabling configuration to provide both image quality and patient tolerance.

### Multi-Flash and Multi-Frame Visible-Light Capture Variants

In some cases, the visible-light capture event comprises a single flash. In other cases, the visible-light capture event comprises a plurality of time-multiplexed sub-flashes delivered within a single capture window, such that the patient perceives the event as one flash (for example, due to temporal integration of human perception), while the image sensor acquires multiple distinct frames. The sub-flashes can be separated by inter-pulse delays selected to coordinate with sensor exposure timing, rolling or global shutter readout constraints, and/or flash driver charging characteristics. The capture window may target less than about 200 ms in some implementations, less than about 100 ms in some implementations, or otherwise select to balance motion robustness, patient comfort, and image quality.

The plurality of sub-flashes can be controlled to differ from one another in one or more respects, including peak intensity, pulse width, duty cycle, spectral content, illumination geometry (for example, selection among multiple LEDs or illumination channels positioned at different angles), polarization state, or relative timing with respect to the sensor exposure window. In certain cases, the system uses a sequence of different exposure settings and/or different flash intensities to obtain a set of frames suitable for high-dynamic-range (HDR) processing or highlight suppression (for example, reducing specular reflection artifacts), while maintaining clinically acceptable total delivered energy. In certain implementations, the system uses multiple lower-energy sub-flashes instead of a single higher-energy flash to reduce blink probability and improve patient comfort while still achieving sufficient signal-to-noise ratio through subsequent fusion or selection.

In some cases, the processing circuitry registers and combines the plurality of captured frames to generate a composite color retinal image. The composite generation can include one or more of: selecting a best frame based on quality scoring, aligning frames using image registration, rejecting frames that exceed motion thresholds, computing a weighted average or robust average, performing deghosting, performing HDR fusion, or locally blending regions from different frames (for example, replacing saturated regions from one frame with unsaturated regions from another frame). In certain cases, the system retains both (i) a composite image and (ii) one or more constituent frames, and the second-stage quality assessor (HQA-2) evaluates the composite and/or the constituent frames to determine whether at least one diagnostically sufficient result has been obtained. If the composite result fails a quality threshold but one constituent frame passes, the system may select and output that constituent frame as the diagnostic image, optionally together with metadata indicating that it was selected from a multi-flash capture burst.

In some cases, the number of sub-flashes, their timing, and/or their intensities are adaptively chosen based on one or more of: measured pupil size, estimated eye or device motion (including motion stability gating), prior capture outcomes, predicted glare risk, ambient illumination, or a patient comfort setting. For example, when motion stability is low, the system may shorten the capture window, reduce the number of frames, or emphasize best-frame selection. When glare risk is high, the system may employ an HDR sequence or vary illumination geometry across sub-flashes. These variations are intended to be illustrative and not limiting, and the visible-light capture event may be implemented with any combination of single-flash, time-multiplexed multi-flash, multi-frame acquisition, best-frame selection, and/or composite formation.

In some instances, the retina camera 300 may employ one or more light sources configured to dynamically switch between IR or NIR and visible light, thus providing enhanced control over the imaging process and reducing the number of components. Switching control may be performed via firmware routines on the ICS controller or, in some configurations, delegated to dedicated hardware timing logic (e.g. FPGA or ISP) for deterministic synchronization with sensor exposure cycles. This configuration offers flexibility in adapting the light sources to specific clinical or operational requirements.

In some implementations, the retinal camera includes a camera control subsystem (implemented in firmware, an ISP, an FPGA, an ASIC, a system-on-chip, or combinations thereof) configured to coordinate illumination, sensor exposure, and readout timing during transitions between IR or NIR guidance imaging and visible-light capture. In some configurations, multi-frame capture gating is implemented as firmware on the controller/CPU and is logically distinct from HQA-1, while in other configurations, gating is performed by dedicated timing logic (such as, ISP, FPGA, or ASIC) that evaluates buffered HQA-1 outputs and motion stability signals. In alternative implementations, HQA-1 itself performs multi-frame validation, and a separate capture gate module is omitted. Thus, the capture gate functionality can be performed by the electronic circuitry, which may include the controller/CPU, the dedicated timing logic, or HQA-1 executing on the hardware accelerator. The camera control subsystem can maintain a flash energy storage element in a ready state (for example, maintaining a flash capacitor charge state or pre-charge level) during IR or NIR imaging so that a capture command can be executed with low latency. The camera control subsystem can execute a deterministic sensor configuration sequence when transitioning from IR or NIR imaging to visible-light capture, including one or more of readout mode switching, gain adjustment, exposure setting, rolling-shutter or global-shutter configuration, binning or region-of-interest selection, and ISP pipeline reconfiguration. The camera control subsystem can further synchronize a flash trigger with a sensor exposure window (or exposure windows for multiple frames) and can apply calibrated timing offsets to account for driver rise time, sensor integration time, and readout latency, thereby enabling acquisition of a visible-light color image within a target latency budget after an HQA-1 capture indication.

The image sensor (or, in some cases, multiple image sensors) can be configured to capture both IR or NIR and visible light images of the retina, enabling pre-capture and post-capture quality assessments for diagnostic imaging. The image sensor can be part of the camera module 310 in FIG. 3. In some cases, the image sensor can have sensitivity across both visible and IR or NIR wavelengths, allowing efficient, dual-spectrum imaging within a single sensor unit. During the pre-capture phase, the image sensor may capture IR or NIR frames either continuously or intermittently, based on the system’s requirements and specific imaging protocols. These IR or NIR frames can be assessed by HQA-1 to determine if conditions meet the criteria for capturing a diagnostic-quality color image. Once HQA-1 confirms optimal imaging conditions, the image sensor can capture a high-resolution color image under visible light. When operating in multi-frame composite mode, the sensor can acquire several frames during the same flash event, which are then combined to generate a composite image. The image sensor can be optimized for rapid data handling to provide low-latency transmission to the GPU-based processing module for real-time processing without compromising image quality.

The image sensor’s processing capabilities may vary based on the system configuration. In some cases, the image sensor includes basic on-sensor processing functions, such as automatic exposure control, gain adjustments, or noise reduction. These preliminary adjustments can help optimize image quality while reducing the processing load on the ICS. In some instances, the image sensor may not perform any on-sensor processing, with all adjustments handled by the electronics before images are passed to HQA-1 or HQA-2. This configuration can allow the ICS to apply custom processing tailored to retinal imaging needs, thus enhancing diagnostic accuracy by optimizing each processing step. In some configurations, the system may employ a combination of basic on-sensor adjustments and more advanced processing in the ICS, balancing initial adjustments with custom processing to achieve optimal diagnostic quality. This flexibility in IR or NIR image capture frequency and processing configuration can allow the system to adapt to various diagnostic imaging requirements, consistently providing high-quality retinal images suitable for HQA-driven assessments.

With reference to FIG. 3, a hardware accelerator or electronics (which can be a GPU-accelerated electronics module) can perform real-time, low-latency processing, enabling the complex operations of the dual-stage HQA (illustrated as HQA-1 342 and HQA-2 344). The electronics can include a local or embedded GPU 340, an ICS controller or controller 330 (which can be a CPU), and memory for storing one or more models and their parameters. Together, these components can support high-speed data handling, inference, and parallel processing capabilities to perform real-time decision-making and comprehensive quality assessments. As used herein, electronic circuitry can include, without limitation the controller, the hardware accelerator (e.g., GPU, NPU, or TPU), and any dedicated timing logic (e.g., ISP, FPGA, or ASIC), any of which can perform parts of the capture control and quality assessment functions described. In some implementations the hardware accelerator may execute HQA-1 continuously during a screening session while the IR or NIR video feed is running. In some power-optimized implementations, the hardware accelerator and/or the HQA-1 processing path is maintained in an inactive or low-power state unless and until a screening session is initiated and/or a capture trigger unlocks HQA-1 execution, and the hardware accelerator and/or the HQA-1 processing path is returned to an inactive or low-power state upon termination of the screening session, cancellation, completion, or timeout. In some power-optimized cases, HQA-1 runs continuously for providing feedback to the user, but it does not trigger the capture of a color image unless a capture trigger is active, which allows providing guidance on-screen without automatically initiating color image capture.

The embedded GPU 340 can accelerate inference processes for HQA’s deep learning models (or, in some cases, machine learning models), enabling complex, holistic quality assessments with minimal latency. The HQA-1 and HQA-2 deep learning models can be optimized and deployed to run efficiently on the GPU hardware. This can allow IR- or NIR-based HQA-1 to process and give results in real-time, and color-based HQA-2 to process and give results with minimal latency. In certain implementations, GPU resources can also be shared with per-alignment and image composite algorithms without disrupting real-time performance. The parallel processing capabilities of the GPU can facilitate analysis of IR or NIR frames in real-time and run multiple quality assessment processes simultaneously, thus facilitating efficient, accurate, and timely evaluations. Deep learning or machine learning models within one or more of HQA-1 or HQA-2 may be pruned, quantized, fused, or otherwise optimized to improve efficiency and reduce power consumption while preserving inference speed, thus making ICS highly suitable for handheld or mobile applications.

Pruning can involve removing unnecessary weights and layers based on their importance. Quantization can involve conversion of floating-point weights or other model variables to lower precision format (such as, integer format) to reduce computational load. Fusion can involve fusing layers to eliminate the need to store intermediate outputs. Optimization can involve preprocessing (such as, resizing or normalization), which can be performed on the GPU 340 (for instance, using Nvidia’s CUDA platform). Model architecture can be optimized for high performance with smaller memory requirements, for instance, using model distillation to train a smaller model.

In some cases, the GPU can support supplementary processes within one or more of HQA-1 or HQA-2, which may include other image processing or machine learning techniques, providing additional data points that may enrich the overall quality assessment process. These supplementary processes can be executed in parallel with the models of one or more of HQA-1 or HQA-2.

In addition to processing, the electronics can execute firmware for low-level device control, an operating system and include memory for storing model parameters and data as well as software optimized for deep learning inference libraries. These components can be structured to enable high-speed data transfer and maintain low-latency operation across the ICS. The operating system or firmware can coordinate interactions between the GPU, HQA, image sensor, and capture functionality, and is configured to provide seamless operation and cohesive functionality within the compact housing of the retina camera. As a result, real-time responsiveness for diagnostic retinal imaging can be achieved.

The controller 330 (sometimes referred to as ICS controller) can provide a comprehensive control framework that manages the entire imaging workflow within the ICS, encompassing pre-capture quality assessment, real-time parameter optimization, image capture, and post-capture quality verification. The controller 330 can be configured to coordinate interactions among components, including light sources, the image sensor, and the GPU 340 executing the dual-stage HQA, which comprises pre-capture quality assessment HQA-1 342 and post-capture diagnostic verification HQA-2 344. By overseeing each phase of the imaging process—from the initial assessment of IR or NIR frames to the final verification of the captured color image—the controller 330 can enable a consistent, high-quality, and low latency imaging process with minimal operator intervention.

As used herein, the controller or ICS controller can refer to the same control component, and references to controller in connection with trigger latching and workflow coordination (such as, with respect to FIG. 1A) can refer to the ICS controller. In certain implementations, the controller maintains the hardware accelerator that runs HQA-1 in an inactive or low-power mode until needed, thereby reducing idle compute, conserving battery, and mitigating thermal load. The controller 330 may be further configured to transition the on-board hardware accelerator and/or an HQA-1 processing path back to an inactive or low-power state responsive to termination of a screening session, responsive to completion of the assessment by HQA-2, and/or after expiration of a timeout period , thereby conserving energy during idle intervals while maintaining low-latency readiness when needed.

In some cases, the controller 330 can integrate both HQA-1 and HQA-2 as inherent functionalities within the control framework, with pre-capture and post-capture assessments operating as coordinated stages within the ICS. In this configuration, the controller 330 can directly govern real-time quality evaluation prior to capture, initiate image capture upon verification of optimal imaging conditions (illustrated by the arrow 332 for IR or NIR image capture and the arrow 334 for color image capture in FIG. 3), and conduct post-capture verification of the diagnostic sufficiency of the captured image. This approach is configured so that the ICS operates as a unified system, managing the entire imaging workflow in a fully integrated manner.

As described herein, when implemented as a module distinct from HQA-1, the capture gate can reside on the controller 330 or on dedicated timing logic and evaluate multi-frame HQA-1 outputs (and optionally motion stability metrics) before authorizing visible-light capture. Alternatively, HQA-1 can incorporate the multi-frame validation and provide an armed indication directly to the controller 330, omitting a separate capture gate.

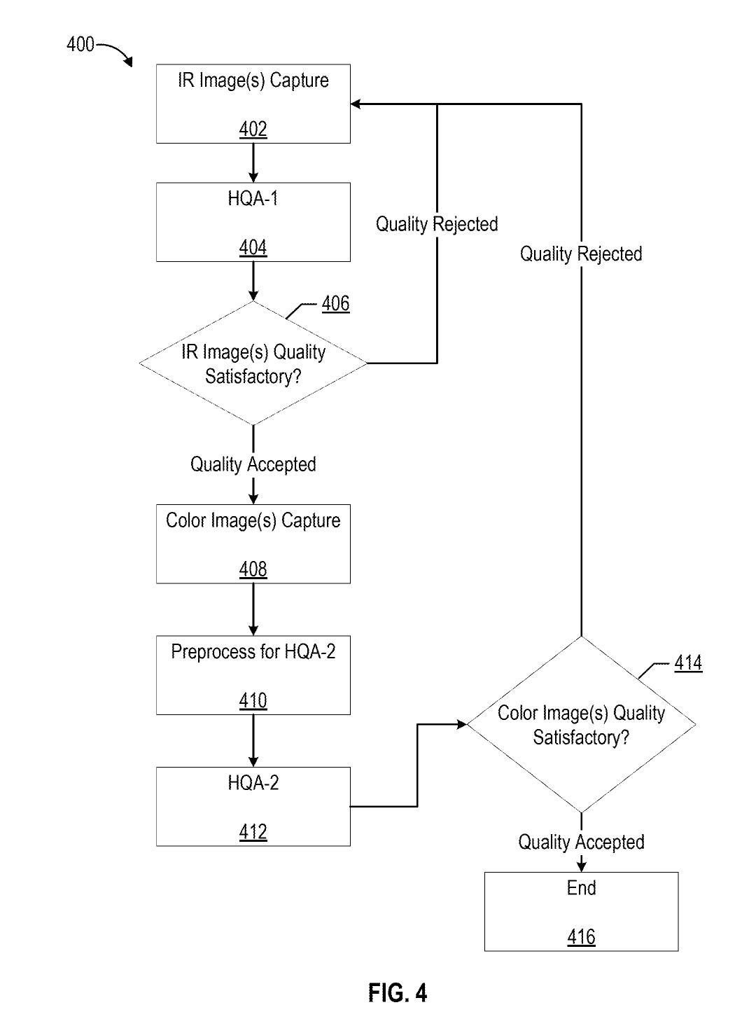

In another approach, the controller 330 can include HQA-1 as an integrated functionality for pre-capture assessment, while HQA-2 can be structured as a separate module or software component for post-capture verification. Even with HQA-2 configured as a distinct module, the controller 330 can retain full coordination over the imaging and quality assessment workflow. The controller 330 can direct HQA-1 to initiate capture based on real-time quality analysis and subsequently can rely on HQA-2 verification results to determine further actions, such as advancing to the next stage of the imaging protocol or reinitiating the imaging cycle for retake if image quality is insufficient (see FIG. 4).

In yet another approach, the controller 330 can be configured to delegate specific processing tasks to dedicated hardware or firmware modules, such as the GPU 340 that runs both HQA-1 and HQA-2, while the controller 330 itself retains central control over decision-making and workflow management. This can allow the controller 330 to leverage specialized processing hardware while maintaining comprehensive control over each stage of the imaging workflow.

Regardless of the specific configuration or distribution of quality assessment components—whether HQA-1 and HQA-2 are implemented as integrated functions, distinct modules, or distributed components—the controller 330 can retain centralized control and coordination over every step of the imaging workflow, including pre-capture assessment, capture initiation, and post-capture verification. This flexibility can be configured so that the ICS can be adapted to various hardware and software configurations while achieving an automated, unified workflow for high-quality diagnostic retinal imaging.

### Dual-Stage Image Quality Assessment

Holistic Quality Assessor (HQA-1) 342 can be configured to perform real-time quality assessment on IR or NIR image frames (illustrated as 352 in FIG. 3) prior to color image capture (illustrated as 354 in FIG. 3). HQA-1 can be executed by the GPU 340. Positioned as the initial stage in a dual-stage assessment process, HQA-1 can evaluate IR or NIR image frames (which can be stored in memory) to determine whether they are likely to yield a diagnostic-quality color image. HQA-1 can include one or more deep learning models (or machine learning models) trained on large datasets (such as, 100,000 or more images) to extract complex features from IR or NIR images especially when dealing with images with varied lighting conditions. In the simplest implementation, HQA-1 can include a single deep learning model trained on labeled IR or NIR retinal images. This model can perform a holistic analysis of each IR or NIR image frame of a plurality of IR or NIR images (which may be captured as a video stream) and, upon identifying a frame of sufficient quality determined based on comparing at least one characteristic of the IR or NIR image frame to at least one threshold, output an indication that the IR or NIR image is of sufficient quality, which would trigger the capture of a color image. This configuration provides an efficient, streamlined solution for identifying optimal frames with minimal computational complexity.

HQA-1 can utilize one or more convolutional neural networks (CNNs). When HQA-1 operates with a single deep learning model for simplicity, HQA-1 can analyze IR or NIR image frames holistically without reliance on discrete parameter evaluations. In some implementations, HQA-1 can integrate machine learning or image processing models to assess specific quality indicators, such as focus sharpness, illumination uniformity, or glare. These models may be executed in parallel with the primary deep learning model, contributing additional data points that enhance the overall accuracy of HQA-1’s assessments. In yet another implementation, HQA-1 may adjust its quality criteria (such as, one or more thresholds) based on preconfigured imaging protocols or user-selected settings, providing an adaptable solution for various diagnostic needs.

In more complex implementations, HQA-1 may employ one or more deep learning models to analyze multidimensional features within the IR or NIR image frames, identifying patterns and qualities that contribute to diagnostic image quality. Unlike existing systems that evaluate individual parameters in isolation, such as focus or brightness, HQA-1 can assess these characteristics holistically within a comprehensive quality framework. The output from HQA-1 may include a confidence score, probability metric, or other quality indicator signaling whether a frame meets one or more quality thresholds for diagnostic use.

Unlike existing systems that evaluate parameters in isolation, HQA-1 can analyze multiple image features or parameters collectively. HQA-1 can analyze one or more of the following parameters of an IR or NIR image:

Retinal coverage: confirms that a sufficient portion of the retina is visible within the image frame.

Focus sharpness: configured to verify that the retina is in focus to capture diagnostic details.

Illumination uniformity: evaluates if IR or NIR exposure is balanced across the field.

Glare detection: identifies overexposed or highly reflective areas that could obscure retinal details.

Contrast and noise levels: configured to verify that the image is neither too dark nor too grainy.

Alignment and centering: determines if the retina is positioned correctly for a full view.

HQA-1 can determine quality of images at least on par with an expert eye specialist. In some instances, the IR or NIR image quality can be insufficient for one reason, such as, bright glare blocking out the retina or the entire image is black. In other instances, it is an amalgamation of things that come together to indicate whether a clinician has confidence that the image is good enough quality to provide a confident assessment of presence of a disease.

Upon determining that one or more quality thresholds have been satisfied, HQA-1 can communicate with the controller 330 (illustrated by the arrow 343 in FIG. 3) to cause initiation of the capture sequence of a color image (illustrated by the arrow 334 in FIG. 3). In systems supporting composite capture, the trigger may initiate a short burst of frames during a visible-light flash, with the controller registering and combining them on-device before HQA-2 analysis. If one or more quality thresholds have not been satisfied but specific parameters could be optimized to improve image quality, HQA-1 may instruct the Real-Time Imaging Optimizer (RIO) 360 to make targeted adjustments, such as refining focus or adjusting illumination. This selective engagement of RIO is configured to modify only the parameters directly affecting image quality are modified, maintaining low latency while increasing the likelihood of capturing a diagnostic-quality image.

To accommodate diverse imaging conditions and computational needs, HQA-1 may operate in various modes. In frame-by-frame assessment mode, HQA-1 can continuously evaluate each IR or NIR video frame in real-time, providing high responsiveness under dynamic imaging conditions. In reduced frame analysis mode, HQA-1 can assess every Nth frame (such as, every second, third, fourth, or the like frame) to lower the computational load, which can minimize latency, power usage, and heat generation. In manual or triggered mode, HQA-1 can initiate the assessment process only upon manual activation by the operator to lower the computational load. In a screening-session mode, initiation of a screening session (for example, by an operator selecting a “Start Screening” control on the user interface, or automatically according to a protocol) may cause activation of IR or NIR guidance imaging, and causes HQA-1 to evaluate IR or NIR frames in real time as the frames arrive. In a power-saving gated mode, IR or NIR frames may be continuously captured and displayed on the user interface, while HQA-1 executes only when a capture trigger unlocks an HQA-1 processing path. In a capture-authorization mode, HQA-1 may execute continuously (for example, to provide user interface feedback) while visible-light flash capture is inhibited unless a capture trigger is active. This mode can reduce idle compute, conserve battery, and mitigate thermal accumulation, with the hardware accelerator otherwise maintained in an inactive or low-power state. In fixed duration mode, HQA-1 can perform continuous assessments for a predetermined period before pausing until the next activation cycle to lower the computational load. These operational modes allow HQA-1 to flexibly adapt to a range of clinical settings and user requirements.

HQA-1’s output may be presented on the user interface in real-time, facilitating provision of live feedback on quality to the operator. This real-time quality feedback can serve as an intuitive guide to assist the operator in positioning and alignment. Through visual cues or a quality indicator on the display, the system can help the operator make adjustments to improve image conditions (such as, center the retina within a field of view) thereby enhancing the likelihood of capturing a diagnostically adequate image. This can be especially beneficial for novice operators, as it reduces the skill required to achieve optimal positioning and timing. For example, the quality indicator may comprise a dot on the display that is red when HQA-1 outputs insufficient quality and green when HQA-1 outputs sufficient quality. In certain implementations, the quality indicator may be driven by the feedback buffer applying temporal smoothing including low-pass filtering (for example, an exponential moving average of a per-frame quality score or probability) and optionally hysteresis and/or debounce logic to reduce frame-to-frame flicker in the displayed quality indicator.

The Real-Time Imaging Optimizer (RIO) 360 can be optionally implemented by the controller 330 or the GPU 340 to perform real-time adjustments to imaging parameters to optimize image quality. RIO can be activated (for instance, by the controller 330 as illustrated by the arrow 362 in FIG. 3) based on feedback from HQA-1 (illustrated by the arrow 343 in FIG. 3), identifying specific deficiencies (or parameters) that can be corrected through targeted parameter optimization, or it may be manually triggered by the operator as needed. RIO may be implemented by the GPU 340 for accelerated processing, enabling rapid adjustments to parameters with minimal latency, which can be particularly beneficial in dynamic imaging scenarios.

HQA-1 and RIO can be executed concurrently. For instance, the GPU 340 can execute HQA-1 while the controller 330 can execute RIO.

In its simplest implementation, RIO adjusts a single parameter, such as focus, to improve image clarity with minimal processing requirements. In more advanced implementations, RIO dynamically adjusts multiple parameters and is configured to provide optimal imaging conditions, particularly in handheld applications where slight movements by the patient or operator can impact alignment, focus, and lighting. By leveraging GPU-accelerated processing, RIO can efficiently manage real-time parameter adjustments, and is configured to improve only those factors directly affecting diagnostic quality, thereby reducing latency and enhancing the overall responsiveness of the ICS 320.

In an example, RIO adjusts focus through motorized control, responding to either HQA-1 feedback indicating suboptimal clarity or manual activation by the operator. Focus adjustments may be implemented via mechanical lens adjustments or automated refocusing processes that interact directly with the camera 310 (such as, with the camera’s firmware). In another example, RIO adjusts light source intensity and exposure settings to address lighting issues detected by HQA-1 or based on operator input. With GPU acceleration, RIO can quickly adjust illumination levels across the retina, minimizing artifacts, such as glare and shadowing, that could interfere with diagnostic interpretation. For example, RIO can decrease the illumination intensity and thus brightness responding to HQA-1 feedback that glare is present in the image. As another example, RIO can increase the illumination intensity responding to HQA-1 feedback that the image lack sufficient contrast (such as, is too dark).

In some implementations, RIO may incorporate other capabilities based on real-time imaging conditions, including adjustments to contrast, aperture settings, or temporal noise reduction. The GPU-accelerated processing can enable these additional capabilities to be performed simultaneously and in real-time, providing flexible options for achieving optimal image quality under diverse clinical conditions.

RIO may operate in multiple modes, with varying levels of responsiveness based on preconfigured settings, specific imaging protocols, or manual operator control. For instance, in a high-responsiveness mode, RIO may continuously monitor and adjust parameters (for example, using the GPU 340 for rapid processing). In a controlled mode, adjustments may occur only upon specific triggers, such as operator command or significant changes in imaging conditions.

RIO’s rapid adjustments can be particularly advantageous in handheld applications, where fast response times are crucial to maintaining optimal alignment, focus, and lighting conditions. RIO is configured to provide maintenance of optimal conditions required to capture high-quality, diagnostic images, with faster and more efficient adjustments that enhance both image quality and system responsiveness.

Holistic Quality Assessor (HQA-2) 344 can provide the second stage of quality assessment, evaluating the captured color image (which can be stored in memory) and is configured to verify that it meets diagnostic standards. In some implementations, using one or more deep learning models specifically trained on color retinal images, HQA-2 assesses the image holistically, analyzing multidimensional features collectively to determine overall diagnostic adequacy. Upon completing its analysis, HQA-2 can provide immediate feedback to the operator via the user interface, enabling informed decision-making regarding image quality. HQA-2 can utilize one or more convolutional neural networks (CNNs)

HQA-2 can be trained on diagnostic-quality labeled color retinal images. For holistic analysis, HQA-2 can analyze one or more following features or parameters of a color image:

Clarity and sharpness: is configured to verify that fine details are visible.

Color balance and contrast: evaluates if the image maintains diagnostic fidelity.

Retinal coverage and positioning: determines whether essential regions are captured.

Presence of artifacts or obstructions: detects issues like shadows, reflections, or motion blur, glare vignetting.

Diagnostic adequacy: assesses whether the image meets clinical requirements for disease identification.

HQA-2 can provide for output to the user one or more of the captured color image, feedback regarding a determination of quality of the captured color image, options for accepting or rejecting the quality determination, or metadata on image quality parameters.

HQA-2 can provide for output to the user a quality rating or pass/fail indicator on the user interface (for instance, a display), allowing the operator to accept, retake, or proceed (for instance, after further review) with the image. If HQA-2 suggests a retake a color image due to insufficient quality, this recommendation may not necessarily imply discarding the original image. Rather, it can serve as guidance to capture an additional image of sufficient quality. This interactive, immediate feedback can allow the operator to decide whether to proceed with the current image, retain it for reference, or capture another image to obtain diagnostic adequacy.

In some implementations, the controller 330 may automatically proceed with the imaging protocol based on HQA-2’s output (illustrated by the arrow 345 in FIG. 3). If HQA-2 confirms diagnostic adequacy, the controller 330 may advance to the next imaging step (such as, obtain additional image(s)). If the quality is insufficient, the controller 330 may notify the user, automatically restart the IR or NIR image capture (for instance, the IR or NIR video feed), and reinitiate the HQA-1 assessment process. This process can enable a continuous cycle of quality evaluation and image capture until the imaging protocol is successfully completed.

In certain cases, image preprocessing may optionally be performed prior to HQA-2’s analysis. Image preprocessing can include adjustments, such as color balance, contrast enhancement, or noise reduction, which optimize the image for HQA-2’s holistic evaluation. Preprocessing can be tailored to align with HQA-2’s diagnostic interpretation criteria, further enhancing overall quality verification.

HQA-2’s output can be used to enhance data labeling for model improvements. When an IR or NIR image is saved alongside its corresponding color image, HQA-2’s quality rating can label the IR or NIR image, indicating whether it led to a diagnostically sufficient color image. This labeled data can then be incorporated into datasets used to retrain or improve HQA-1’s performance, refining its ability to predict image quality accurately.

In some implementations, for optimal performance and efficiency, at least one model within HQA-2 can be executed by the GPU 340, thereby leveraging GPU-accelerated processing to execute image quality assessment with minimal latency. This can enable HQA-2 to provide real-time feedback to the operator and enhance overall system responsiveness, particularly in environments where rapid image assessment is required. GPU acceleration can also support the efficient operation of other deep learning and supplementary algorithms in HQA-2, configured to support consistent capture of diagnostically sufficient images with minimal delay. Through this final, holistic quality verification, HQA-2 is configured to verify that images suitable for diagnostic interpretation are captured reliably, thus supporting an efficient and robust retinal imaging workflow.

### Dual-Stage Image Quality Assessment Workflow

The workflow of the ICS 320 for retinal imaging can be structured to capture high-quality diagnostic images with minimal operator intervention through an integrated sequence of assessments, parameter adjustments, and image capture operations, all coordinated by the controller 330. FIG. 4 illustrates an example workflow as an image capturing process 400, which can be implemented by the retina camera 300 (such as, by the controller 330 and GPU 340). With reference to FIG. 4, the workflow can include one or more of the following.

System Initialization: Upon powering on, the controller 330 can initialize, for instance, the camera 310 (including light sources and image sensor), GPU 340, and the user interface.

Pre-capture IR or NIR Imaging: After initialization, in block 402 the process 400 can activate one or more IR or NIR light sources, which may provide continuous, non-invasive illumination to allow retinal imaging without the need for pharmacological pupil dilation. The image sensor can begin capturing IR or NIR image frames, either continuously or intermittently, according to system requirements and imaging protocols. IR or NIR image frames can be captured as a video stream. In some implementations, activation of IR or NIR guidance imaging may occur responsive to initiation of a screening session (for example, via an operator selecting a “Start Screening” control on the display), and the IR or NIR video feed may run continuously during the screening session with frames streamed to the user interfaces and provided, in parallel, to HQA-1 in real time. In alternative implementations, IR or NIR capture may continue for display while HQA-1 is selectively enabled by a capture trigger, and/or visible-light flash capture is inhibited unless a capture trigger is active. Upon termination of the screening session, cancellation, completion, or timeout, HQA-1 and/or the hardware accelerator can be paused or transitioned to an inactive or low-power state to reduce power consumption and thermal load.

Pre-Capture Quality Assessment by HQA-1: The process 400 can transition to block 404 where the IR or NIR image frames are processed in real-time by HQA-1, which can perform holistic analysis of each frame. HQA-1 can assess the IR or NIR image frames to determine if they meet the criteria likely to yield a diagnostic-quality color image. The evaluation may be conducted for two purposes – updating the quality indicator in the feedback buffer for operator guidance, and populating the automated capture buffer with consecutive sufficient quality frames that meet stricter gating thresholds for triggering capture. In some implementations, the feedback buffer and the automated capture buffer may be implemented using a same rolling history of HQA-1 outputs, with temporal smoothing used for operator guidance and multi-frame validation over the rolling history used to gate capture (for example, requiring sufficient quality for more than one frame captured at different times within a rolling time window and/or for a configurable run of consecutive frames). In certain implementations, the feedback buffer may apply temporal smoothing including low-pass filtering, for example by applying an exponential moving average to per-frame HQA-1 quality outputs, optionally with hysteresis and/or debounce logic to stabilize the quality indicator despite frame-to-frame fluctuations. For example, the quality indicator may comprise a dot displayed on the user interface that is red when HQA-1 outputs insufficient quality and green when HQA-1 outputs sufficient quality. HQA-1 may operate in various modes (such as, frame-by-frame, reduced frame analysis, triggered, or fixed-duration) to balance responsiveness with computational efficiency based on the operational requirements. In implementations employing a distinct capture gate, the capture gate is executed on the controller or dedicated timing logic and consumes HQA-1 quality outputs. Alternatively, HQA-1 can internally perform multi-frame validation and provide an armed indication directly to the ICS controller.

RIO: Optionally (and not illustrated in FIG. 4), if HQA-1 identifies deficiencies that may be resolved through parameter adjustments, Real-Time Imaging Optimizer (RIO) can be executed to modify imaging parameters, such as focus, light source intensity, or exposure settings. RIO can be executed by the GPU 340 for accelerated processing, enabling rapid, real-time adjustments with minimal latency. RIO’s adjustments can optimize only the parameters crucial for achieving diagnostic quality, which can be especially advantageous for handheld applications where imaging conditions change dynamically.

HQA-1 Output: In block 406, the process 400 can determine whether the quality of the IR or NIR image frame is acceptable. Once HQA-1 detects that the IR or NIR image frame quality meets one or more thresholds and the automated capture buffer confirms a sufficient run of stable frames, the process 400 can proceed to block 408 to perform color image capture. In certain cases, this capture may consist of a burst of closely timed frames during a single or multiple visible-light flash, which are later combined on -device into a composite image before HQA-2 evaluation. In certain implementations, progression to block 408 is additionally conditioned on a capture authorization input being active (for example, through a capture trigger as described herein), such that HQA-1 may provide user interface guidance without automatically initiating visible-light flash capture unless and until capture is authorized.

If in block 406 any issues are detected that cannot be corrected with parameter adjustments, the process 400 can transition to block 402 and continue with the analysis of IR or NIR image frames. In some cases, the process 400 can notify the operator to make manual adjustments (such as, moving the retina camera).

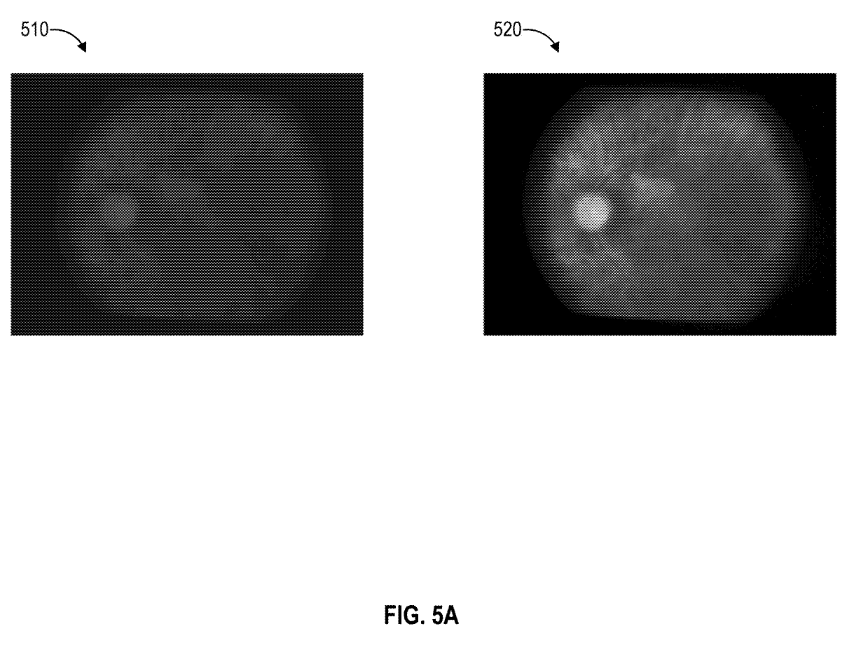

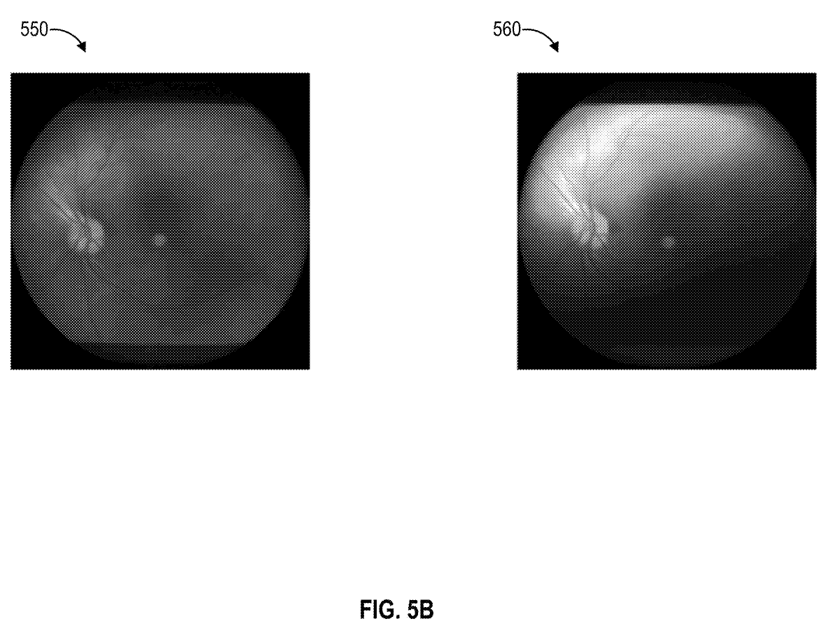

FIG. 5A illustrates an IR or NIR image 510 of unacceptable quality (such as, out of focus and under exposed) and an IR or NIR image 520 of acceptable quality. In some cases, image 520 has been obtained after RIO adjustments.

Image Capture with Visible Light: Once the IR or NIR image of sufficient quality has been captured, the process 400 can initiate capture of a color image. To provide optimal quality for the color image, focus, exposure, and other imaging parameters for are determined based on the IR image’s characteristics. Additionally, the required latency to achieve these settings and capture the color image can be calculated. Latency between capture of IR or NIR image and color image can be less than 100 msec. The process 400 can strategically time the transition from IR or NIR to color image capture to achieve the best possible focus and exposure. In block 408, the process 400 can activate the visible light source(s) to emit a brief, calibrated flash. This visible flash is configured to maintain high-resolution color capture under optimal lighting conditions, with the image sensor capturing a color retinal image for diagnostic purposes. Capture of IR or NIR images can be paused for the purposes of capturing the color image.

HQA-2: Post-Capture Quality Verification: The process 400 can transition to block 410 and perform optional pre-processing prior to HQA-2 analysis. In block 410, pre-processing adjustments (such as, color balance, contrast, or noise reduction) may be applied to the color image before HQA-2 analysis to optimize the image quality for evaluation. Either after performing the pre-processing in block 410 or after capturing the color image in block 408, the process 400 can transition to block 412 where the color image is processed by HQA-2. HQA-2 can perform a holistic evaluation using one or more deep learning models trained on color retinal images. At least one model of HQA-2 can run on the GPU 340, enabling real-time inference and configured to provide minimal latency. In composite mode, HQA-2 may evaluate both the final combined output and individual constituent frames, allowing the system to preserve a single acceptable frame if the composite result falls short of the quality threshold. HQA-2 can assess the color image to verify overall diagnostic adequacy and provide immediate feedback to the operator via the user interface.

FIG. 5B illustrates a color image 560 of unacceptable quality (such as, uneven illumination) and a color image 550 of acceptable quality. These examples may represent single frame or composite results.

Feedback and Completion: If the color image meets diagnostic standards in block 414, the process 400 can determine that the image is adequate for diagnostic interpretation. The process 400 can automatically proceed to the next imaging step (if part of a multi-step imaging protocol) or terminate in block 416. For instance, upon capturing a diagnostically sufficient color image, the process 400 can terminate in block 416 or prepare for the next image in the sequence as part of a comprehensive imaging protocol. Images can be saved according to protocol, and the operator can be provided with a summary of the results on the user interface. In some cases, to optimize storage capacity, the process 400 can store only those color images that have been determined to be of diagnostic quality. The process 400 can do the same for storing any IR or NIR images.

If the image does not meet quality criteria in block 414, the process can transition to block 402 where another IR or NIR image is captured (in some cases, capture of IR or NIR images can be paused during capture of the color image). In some instances, this transition may not be automatic. The process 400 may suggest a retake to the operator (for instance, via the user interface). This recommendation may allow the operator to capture an additional image while retaining the original image if desired. The retake suggestion may include contextual guidance such as “alignment” or “illumination” issues based on HQA-2’s analysis and HQA-1’s capture time metrics, giving the operator a clearer corrective action. In response to operator command, the process 400 may restart the IR or NIR video feed and reinitiate the HQA-1 assessment process to guide the operator toward capturing a better image.

In some instances, the process 400 can automatically prompt a reevaluation cycle based on HQA-2’s output, resuming IR or NIR capture and HQA-1 assessments until an image of sufficient quality is captured.

Data Labeling for HQA-1 Improvement: In some cases (not shown), HQA-2’s output can be used to label corresponding IR or NIR images for dataset creation. By storing both the IR or NIR image and the corresponding color image’s diagnostic adequacy rating, the process 400 can build datasets to retrain and improve HQA-1, refining its predictive capabilities based on prior assessments. In a system with capture, the metadata may include motion stability scores, frame-by-frame quality ratings and fusion success metrics, enabling more accurate model or algorithm development for image composite.

Throughout the workflow, the GPU-accelerated electronics of the retina camera 300 can enable real-time analysis and decision-making by handling data-intensive tasks, running deep learning inferences for both HQA-1 and HQA-2, and supporting supplementary algorithms. The controller 330 can coordinate every stage of this workflow, from pre-capture adjustments to post-capture quality verification and is configured to provide an integrated, automated process that minimizes operator effort while maximizing image quality and diagnostic reliability.

### CONCLUSION

Approaches described herein can optimize image capture utilizing two-stage holistic quality assessment. At the first stage, HQA-1 can perform real-time quality assessments on IR or NIR image frames to determine whether they are likely to yield diagnostically sufficient color images. Unlike existing approaches that analyze specific parameters in isolation, HQA-1 can holistically assess IR or NIR image frames. This approach can capture complex interdependencies among image characteristics, providing a comprehensive quality evaluation. When implemented with a dual-buffer design, HQA-1’s decisions for operator feedback and capture gating can be decoupled, configured to provide a smooth preview experience while maintaining strict stability and quality conditions for capture. By using GPU-accelerated processing, HQA-1 can maintain ultra-low latency (such as, less than 50 msec), enabling color image capture at the precise moment when conditions are ideal. In some cases, the latency of capturing the color image can be less than 50 msec.

After capturing a color image, at the second stage, HQA-2 can perform a post-capture assessment to confirm its diagnostic adequacy. The latency of HQA-2 processing can be less than 100 msec. This immediate quality verification can provide on-screen feedback to the operator, indicating whether to retain (for instance, after further review) or retake the image without discarding the original. HQA-2’s output can label corresponding IR or NIR images for training dataset creation, enabling continuous refinement of HQA-1’s performance. This dual-stage approach is configured to provide high-quality output while minimizing the need for operator judgment or intervention.

Low latency AI inference described herein may be required to be performed on-board any retinal camera disclosed herein without involving one or more external computing devices. For example, the latency demand of receiving the AI inference output signal may be 200 msec or less, which may make it not possible to transmit data to an external computing device (such as, remote server in a data center) while still meeting the latency requirement

In some cases, RIO can be implemented to adjust imaging parameters, such as focus and illumination, based on HQA-1’s feedback. RIO’s GPU-accelerated, selective adjustments can optimize only those parameters most critical to image quality, preserving low latency and enhancing responsiveness in dynamic environments. This approach can be especially valuable in handheld applications, where quick adjustments are essential to maintaining image quality.

Advantageously, a cohesive and highly effective imaging process can be obtained. Real-time coordination of quality assessment, parameter adjustments, and image capture can provide an automated, end-to-end workflow that optimizes each capture, and is configured to provide consistent performance even with limited operator expertise.

Approaches described herein can perform better than an experienced clinician at least due to having a higher capture success rate per attempt, shorter time required per successful image capture, and smaller percentage of images requiring retakes (for instance, by reducing human variability and errors). By integrating motion stability gating, dual-buffer capture control, and optional composite image formation, the system further improves reliability in challenging handheld scenarios where even expert operators may struggle.

In some instances, multiple GPUs can be utilized for parallel processing. While certain examples have been described in the context of capturing retinal images, the approaches described herein can be used for capturing other images, such as those of the ear or skin.

In some implementations, at least some of the processing can be performed externally, for instance, by transmitting one or more of IR or NIR or color images to one or more external computing devices for analysis. In some cases, at least some processing can be performed by one or more external computing device in parallel with on-board processing.

Any of the implementations described herein can utilize one or more features described in one or more of U.S. Patent No. 11,950,847, U.S. Patent No. 12,178,392, U.S. Patent Publication No. 2022/0405927, U.S. Patent Publication No. 2025/0022599, or U.S. 2025/0037277, each of which is incorporated by reference in its entirety.

### Example Implementations

Examples of the implementations of the present disclosure are described in view of the following example clauses. The features recited in the below example implementations are combinable with additional features disclosed herein. Furthermore, additional inventive combinations of features are disclosed herein, which are not specifically recited in the below example implementations, and which do not include the same features as the specific implementations below. For sake of brevity, the below example implementations do not identify every inventive aspect of this disclosure. The below example implementations are not intended to identify key features or essential features of any subject matter described herein. Any of the example clauses below, or any features of the example clauses, are combinable with any one or more other example clauses, or features of the example clauses or other features of the present disclosure.

Clause 1. A retinal camera comprising: a housing; at least one infrared (IR) or near infrared (NIR) light source supported by the housing and configured to provide continuous, non-mydriatic illumination for capturing IR or NIR retinal images of an eye; at least one visible light source supported by the housing and configured to provide flash illumination for capturing color retinal images of the eye; at least one image sensor supported by the housing and configured to capture the IR or NIR retinal images and color retinal images; a user interface supported by the housing; and on-board processing circuitry supported by the housing and comprising at least one processor and a hardware accelerator, the on-board processing circuitry configured to: implement a first Holistic Quality Assessor (HQA-1) comprising at least one first machine learning model that is configured to be executed on the hardware accelerator, the at least one first machine learning model trained on a dataset that includes IR or NIR retinal images, the HQA-1 being configured to: 1) perform real-time quality assessments of the IR or NIR retinal images captured by the at least one image sensor by determining whether at least one property of the IR or NIR retinal images satisfies a first quality threshold and 2) generate quality assessments for a plurality of IR or NIR retinal images; generate an indication that a color retinal image should be captured based on the first quality threshold being satisfied for at least two IR or NIR retinal images captured at different times within a time window; responsive to the indication that the color retinal image should be captured, cause activation of the at least one visible light source to emit one or more flash illumination events during an acquisition interval and cause capture of one or more color retinal image frames by the at least one image sensor; generate an output color retinal image by selecting one of the one or more color retinal image frames or by combining at least two of the one or more color retinal image frames; implement a second Holistic Quality Assessor (HQA-2) comprising at least one second machine learning model that is configured to be executed on the hardware accelerator, the at least one second machine learning model trained on a dataset that includes color retinal images, the HQA-2 configured to: 1) determine whether at least one property of the output color retinal image satisfies a second quality threshold and 2) generate an indication that the output color retinal image is of diagnostic quality responsive to determining that the at least one property satisfies the second quality threshold; and responsive to generating the indication that the output color retinal image is of diagnostic quality, cause the user interface to provide a notification that the color retinal image of diagnostic quality has been captured, wherein the quality assessments performed by the HQA-1 and HQA-2 are performed locally by the on-board processing circuitry without transmitting any of the IR or NIR retinal images or any of the color retinal images to an external computing device for quality assessment.

Clause 2. The retinal camera of clause 1, wherein the on-board processing circuitry is further configured to implement a capture gate configured to determine that the first quality threshold has been satisfied for a threshold number of consecutive or non-consecutive IR or NIR retinal images within the time window prior to activation of the at least one visible light source.

Clause 3. The retinal camera of any one of clauses 1 to 2, wherein the at least one visible light source is configured to emit a plurality of time-multiplexed sub-flashes within a capture window while the at least one image sensor captures multiple distinct color retinal image frames, at least two color retinal image frames being acquired with different exposure parameters.

Clause 4. The retinal camera of clause 3, wherein plurality of time-multiplexed sub-flashes differ in at least one of: peak intensity, pulse width, spectral content, polarization state, or illumination geometry being adaptively selected based on at least one of: measured pupil size, a motion stability metric, ambient illumination, prior capture outcomes, or predicted glare risk.

Clause 5. The retinal camera of any one of clauses 1 to 4, wherein the on-board processing circuitry is further configured to: cause capture of the one or more color retinal image frames further responsive to receiving a capture request being generated as a result of an operator manipulating the user interface; responsive to receiving the capture request, latch the capture request so that the capture request remains asserted until the indication that the output color retinal image is of diagnostic quality has been generated; and cancel the capture request responsive to a cancellation by the operator through the user interface or a timeout.

Clause 6. The retinal camera of clause 5, wherein the on-board processing circuitry is further configured to: maintain the hardware accelerator in an inactive state so that power consumption is reduced; transition the hardware accelerator to an active state and execute the HQA-1 responsive to receiving the capture request, wherein power consumption in the active state is greater than power consumption in the inactive state; and transition the hardware accelerator to the inactive state responsive to completion of the quality assessment by the HQA-2 or a timeout.

Clause 7. The retinal camera of any one of clauses 1 to 6, wherein the on-board processing circuitry is further configured to: responsive to the indication that the color retinal image should be captured, automatically cause activation of the at least one visible light source and capture of the one or more color retinal image frames.

Clause 8. The retinal camera of any one of clauses 1 to 7, wherein the on-board processing circuitry is further configured to: responsive to the first quality threshold not being satisfied, adjust at least one imaging parameter of the at least one IR or NIR light source, the at least one imaging parameter comprising focus, IR or NIR light intensity, or exposure setting.

Clause 9. The retinal camera of any one of clauses 1 to 8, wherein the on-board processing circuitry is further configured to: cause the user interface to output an indication to retake a color retinal image responsive to not generating the indication that the output color retinal image is of diagnostic quality, the indication to retake identifying at least one quality deficiency comprising alignment, illumination, non-uniformity, motion blur, glare, or insufficient retinal coverage.

Clause 10. The retinal camera of any one of clauses 1 to 9, wherein the on-board processing circuitry is further configured to: determine a motion stability metric using at least one motion estimate determined from IR or NIR retinal images and measurements from at least motion sensor; and cause activation of the at least one visible light source further responsive to the motion stability metric satisfying a stability threshold.

Clause 11. The retinal camera of any one of clauses 1 to 10, wherein the on-board processing circuitry is further configured to: implement a feedback buffer that facilitates application of temporal smoothing to the quality assessments by the HQA-1 for providing feedback of IR or NIR retinal image capture on the user interface; and implement an automated capture buffer that facilitates enforcing of multi-frame validation prior to causing activation of the at least one visible light source.

Clause 12. A non-transitory computer readable medium storing instructions that, when executed by a processing circuitry comprising at least one processor and a hardware accelerator, cause the processing circuitry to: implement a first Holistic Quality Assessor (HQA-1) comprising at least one first machine learning model that is configured to be executed on the hardware accelerator, the at least one first machine learning model trained on a dataset that includes infrared (IR) or near infrared (NIR) retinal images, the HQA-1 being configured to: 1) perform real-time quality assessments of IR or NIR retinal images of an eye captured by at least one image sensor when at least one IR or NIR light source provides continuous, non-mydriatic illumination by determining whether at least one property of the IR or NIR retinal images of the eye satisfies a first quality threshold and 2) generate quality assessments for a plurality of IR or NIR retinal images of the eye; generate an indication that a color retinal image should be captured based on the first quality threshold being satisfied for at least two IR or NIR retinal images of the eye captured at different times within a time window; responsive to the indication that the color retinal image should be captured, cause activation of at least one visible light source to emit one or more flash illumination events during an acquisition interval and cause capture of one or more color retinal image frames by the at least one image sensor; generate an output color retinal image by selecting one of the one or more color retinal image frames or by combining at least two of the one or more color retinal image frames; implement a second Holistic Quality Assessor (HQA-2) comprising at least one second machine learning model that is configured to be executed on the hardware accelerator, the at least one second machine learning model trained on a dataset that includes color retinal images, the HQA-2 configured to: 1) determine whether at least one property of the output color retinal image satisfies a second quality threshold and 2) generate an indication that the output color retinal image is of diagnostic quality responsive to determining that the at least one property satisfies the second quality threshold; and responsive to generating the indication that the output color retinal image is of diagnostic quality, to provide a notification that the color retinal image of diagnostic quality has been captured, wherein the quality assessments performed by the HQA-1 and HQA-2 are performed locally by the processing circuitry without transmitting any of the IR or NIR retinal images or any of the color retinal images to an external computing device for quality assessment.

Clause 13. The computer readable medium of clause 12, wherein the processing circuitry is further caused to implement a capture gate configured to determine that the first quality threshold has been satisfied for a threshold number of consecutive or non-consecutive IR or NIR retinal images within the time window prior to activation of the at least one visible light source.

Clause 14. The computer readable medium of any one of clauses 12 to 13, wherein the at least one visible light source is configured to emit a plurality of time-multiplexed sub-flashes within a capture window while the at least one image sensor captures multiple distinct color retinal image frames, at least two color retinal image frames being acquired with different exposure parameters.

Clause 15. The computer readable medium of any one of clauses 12 to 14, wherein the processing circuitry is further caused to: cause capture of the one or more color retinal image frames further responsive to receiving a capture request being generated as a result of an operator manipulating a user interface; responsive to receiving the capture request, latch the capture request so that the capture request remains asserted until the indication that the output color retinal image is of diagnostic quality has been generated; and cancel the capture request responsive to a cancellation by the operator through the user interface or a timeout.

Clause 16. The computer readable medium of clause 15, wherein the processing circuitry is further caused to: maintain the hardware accelerator in an inactive state so that power consumption is reduced; transition the hardware accelerator to an active state and execute the HQA-1 responsive to receiving the capture request, wherein power consumption in the active state is greater than power consumption in the inactive state; and transition the hardware accelerator to the inactive state responsive to completion of the quality assessment by the HQA-2 or a timeout.

Clause 17. The computer readable medium of any one of clauses 12 to 16, wherein the processing circuitry is further caused to: responsive to the indication that the color retinal image should be captured, automatically cause activation of the at least one visible light source and capture of the one or more color retinal image frames.

Clause 18. The computer readable medium of any one of clauses 12 to 17, wherein the processing circuitry is further caused to: responsive to the first quality threshold not being satisfied, adjust at least one imaging parameter of the at least one IR or NIR light source, the at least one imaging parameter comprising focus, IR or NIR light intensity, or exposure setting.

Clause 19. The computer readable medium of any one of clauses 12 to 18, wherein the processing circuitry is further caused to: cause a user interface to output an indication to retake a color retinal image responsive to not generating the indication that the output color retinal image is of diagnostic quality, the indication to retake identifying at least one quality deficiency comprising alignment, illumination, non-uniformity, motion blur, glare, or insufficient retinal coverage.

Clause 20. The computer readable medium of any one of clauses 12 to 19, wherein the processing circuitry is further caused to: determine a motion stability metric using at least one motion estimate determined from IR or NIR retinal images and measurements from at least motion sensor; and cause activation of the at least one visible light source further responsive to the motion stability metric satisfying a stability threshold.

Clause 21. A retinal camera comprising: a housing; at least one infrared (IR) or near infrared (NIR) light source supported by the housing and configured to provide continuous, non-mydriatic illumination for capturing IR or NIR retinal images of an eye; at least one visible light source supported by the housing and configured to provide flash illumination for capturing color retinal images of the eye; at least one image sensor supported by the housing and configured to capture the IR or NIR retinal images and color retinal images; a user interface supported by the housing; and a processing circuitry supported by the housing and comprising at least one processor and a hardware accelerator, the processing circuitry configured to: with at least one first machine learning model that is configured to be executed on the hardware accelerator and trained on a dataset that includes IR or NIR retinal images, perform quality assessments of a plurality of IR or NIR retinal images captured by the at least one image sensor by determining whether at least one property of the plurality of IR or NIR retinal images satisfies a first quality threshold; responsive to determining that the first quality threshold has been satisfied for at least two IR or NIR retinal images captured at different times within a time window, cause activation of the at least one visible light source and capture of a color retinal image by the at least one image sensor; with at least one second machine learning model that is configured to be executed on the hardware accelerator and trained on a dataset that includes color retinal images, determine that at least one property of the color retinal image satisfies a second quality threshold; and responsive to determining that the at least one property of the color retinal image satisfies the second quality threshold, cause the user interface to provide a notification that the color retinal image of diagnostic quality has been captured.

Clause 22. The retinal camera of clause 21, wherein the quality assessments performed by the at least one first and second machine learning models are performed locally by the processing circuitry without transmitting any IR or NIR retinal images or any color retinal images to an external computing device for quality assessment.

Clause 23. The retinal camera of any one of clauses 21 to 22, wherein the processing circuitry is further configured to determine that the first quality threshold has been satisfied for a threshold number of consecutive or non-consecutive IR or NIR retinal images within the time window prior to activation of the at least one visible light source.

Clause 24. The retinal camera of any one of clauses 21 to 23, wherein: the at least one visible light source is configured to emit a plurality of time-multiplexed sub-flashes within a capture window while the at least one image sensor captures a plurality of color retinal image frames, at least two color retinal image frames being acquired with different exposure parameters; and the processing circuitry is configured to generate the color retinal image by selecting one of the plurality of color retinal image frames or by combining at least two of the plurality of color retinal image frames.

Clause 25. The retinal camera of clause 24, wherein plurality of time-multiplexed sub-flashes differ in at least one of: peak intensity, pulse width, spectral content, polarization state, or illumination geometry being adaptively selected based on at least one of: measured pupil size, a motion stability metric, ambient illumination, prior capture outcomes, or predicted glare risk.

Clause 26. The retinal camera of any one of clauses 21 to 25, wherein the processing circuitry is further configured to: cause capture of the color retinal image further responsive to receiving a capture request being generated as a result of an operator manipulating the user interface; responsive to receiving the capture request, latch the capture request so that the capture request remains asserted until it has been determined that the at least one property of the color retinal image satisfies the second quality threshold; and cancel the capture request responsive to a cancellation by the operator through the user interface or a timeout.

Clause 27. The retinal camera of clause 26, wherein the processing circuitry is further configured to: maintain the hardware accelerator in an inactive state so that power consumption is reduced; transition the hardware accelerator to an active state and execute the at least one first machine learning model responsive to receiving the capture request, wherein power consumption in the active state is greater than power consumption in the inactive state; and transition the hardware accelerator to the inactive state responsive to completion of color image quality assessment by the at least one second machine learning model or a timeout.

Clause 28. The retinal camera of any one of clauses 21 to 27, wherein the processing circuitry is further configured to: responsive to determining that the first quality threshold has been satisfied, automatically cause activation of the at least one visible light source and capture of the color retinal image.

Clause 29. The retinal camera of any one of clauses 21 to 28, wherein the processing circuitry is further configured to: responsive to determining that the first quality threshold has not being satisfied, adjust at least one imaging parameter of the at least one IR or NIR light source, the at least one imaging parameter comprising focus, IR or NIR light intensity, or exposure setting.

Clause 30. The retinal camera of any one of clauses 21 to 29, wherein the processing circuitry is further configured to: cause the user interface to output an indication to retake a color retinal image responsive to not determining that the at least one property of the color retinal image satisfies the second quality threshold.

Clause 31. The retinal camera of clause 30, wherein the indication to retake identifies at least one deficiency comprising alignment, illumination, non-uniformity, motion blur, glare, or insufficient retinal coverage.

Clause 32. The retinal camera of any one of clauses 21 to 31, wherein the processing circuitry is further configured to: determine a motion stability metric using at least one motion estimate determined from IR or NIR retinal images and measurements from at least motion sensor; and cause activation of the at least one visible light source further responsive to the motion stability metric satisfying a stability threshold.

Clause 33. The retinal camera of any one of clauses 21 to 32, wherein the processing circuitry is further configured to: implement a feedback buffer that facilitates application of temporal smoothing to the quality assessments by the at least one first machine learning model for providing feedback of IR or NIR retinal image capture on the user interface; and implement an automated capture buffer that facilitates enforcing of multi-frame validation prior to causing activation of the at least one visible light source.

Clause 34. The retinal camera of any one of clauses 21 to 33, wherein the at least one property of the plurality of IR or NIR retinal images comprises one or more of: retinal coverage, focus, illumination uniformity, glare detection, contrast, or retinal alignment and centering.

Clause 35. The retinal camera of any one of clauses 21 to 34, wherein the at least one property of the color retinal image comprises one or more of: clarity and sharpness, color balance and contrast, retinal coverage, presence of artifacts, or diagnostic adequacy of disease identification.

Clause 36. A non-transitory computer readable medium storing instructions that, when executed by a processing circuitry comprising at least one processor and a hardware accelerator, cause the processing circuitry to execute any of the clauses 21-35.

Clause 37. A method of operating the retinal camera of any one of the preceding clauses.

Clause 38. A retinal camera comprising: at least one infrared (IR) or near infrared (NIR) light source configured to provide continuous, non-mydriatic illumination for capturing IR or NIR retinal images of an eye; at least one visible light source configured to provide flash illumination for capturing color retinal images of the eye; at least one image sensor configured to capture the IR or NIR retinal images and color retinal images; and an electronic processing circuitry configured to: with at least one first machine learning model, perform a first quality assessment of at least one IR or NIR retinal image; responsive to a positive first quality assessment, cause activation of the at least one visible light source and capture of a color retinal image; with at least one second machine learning model, perform a second quality assessment of the color retinal image; and responsive to a positive second quality assessment, provide a notification that the color retinal image of diagnostic quality has been captured.

Clause 39. The retinal camera of clause 38, wherein the first and second quality assessments performed by the at least one first and second machine learning models are performed locally by the electronic processing circuitry without transmitting any IR or NIR retinal images or any color retinal images to an external computing device for quality assessment.

Clause 40. The retinal camera of any one of clauses 38 to 39, wherein the electronic processing circuitry is further configured to perform the first quality assessment for a plurality of IR or NIR retinal images.

Clause 41. The retinal camera of any one of clauses 38 to 40, wherein the at least one visible light source is configured to emit a plurality of time-multiplexed sub-flashes within a capture window while the at least one image sensor captures a plurality of color retinal image frames, at least two color retinal image frames being acquired with different exposure parameters.

Clause 42. The retinal camera of clause 41, wherein the electronic processing circuitry is configured to generate the color retinal image by selecting one of the plurality of color retinal image frames or by combining at least two of the plurality of color retinal image frames.

Clause 43. The retinal camera of any one of clauses 41 to 42, wherein plurality of time-multiplexed sub-flashes differ in at least one of: peak intensity, pulse width, spectral content, polarization state, or illumination geometry being adaptively selected based on at least one of: measured pupil size, a motion stability metric, ambient illumination, prior capture outcomes, or predicted glare risk.

Clause 44. The retinal camera of any one of clauses 38 to 43, wherein the electronic processing circuitry is further configured to cause capture of the color retinal image further responsive to receiving a capture request.

Clause 45. The retinal camera of clause 44, wherein the electronic processing circuitry is further configured to: responsive to receiving the capture request, latch the capture request so that the capture request remains asserted until occurrence of the positive second quality assessment; and cancel the capture request responsive to a cancellation by an operator or a timeout.

Clause 46. The retinal camera of any one of clauses 44 to 45, wherein the electronic processing circuitry is further configured to: maintain a hardware accelerator of the electronic processing circuitry in an inactive state so that power consumption is reduced; transition the hardware accelerator to an active state and execute the at least one first machine learning model responsive to receiving the capture request, wherein power consumption in the active state is greater than power consumption in the inactive state; and transition the hardware accelerator to the inactive state responsive to completion of color image quality assessment by the at least one second machine learning model or a timeout.

Clause 47. The retinal camera of any one of clauses 38 to 46, wherein the electronic processing circuitry is further configured to: responsive to the positive first quality assessment, automatically cause activation of the at least one visible light source and capture of the color retinal image.

Clause 48. The retinal camera of any one of clauses 38 to 47, wherein the electronic processing circuitry is further configured to: responsive to the first quality assessment, adjust at least one imaging parameter of the at least one IR or NIR light source, the at least one imaging parameter comprising focus, IR or NIR light intensity, or exposure setting.

Clause 49. The retinal camera of any one of clauses 38 to 48, wherein the electronic processing circuitry is further configured to: output an indication to retake a color retinal image responsive a negative second quality assessment.

Clause 50. The retinal camera of clause 49, wherein the indication to retake identifies at least one deficiency comprising alignment, illumination, non-uniformity, motion blur, glare, or insufficient retinal coverage.

Clause 51. The retinal camera of any one of clauses 38 to 50, wherein the electronic processing circuitry is further configured to: determine a motion stability metric using at least one motion estimate determined from IR or NIR retinal images and measurements from at least motion sensor; and cause activation of the at least one visible light source further responsive to the motion stability metric satisfying a stability threshold.

Clause 52. The retinal camera of any one of clauses 38 to 51, wherein the electronic processing circuitry is further configured to: implement a feedback buffer that facilitates application of temporal smoothing to a plurality of quality assessments by the at least one first machine learning model for providing feedback of IR or NIR retinal image capture; and implement an automated capture buffer that facilitates enforcing of multi-frame validation prior to causing activation of the at least one visible light source.

Clause 53. The retinal camera of any one of clauses 38 to 52, wherein the first quality assessment of the at least one IR or NIR retinal image comprises analyzing at least one property of the at least one IR or NIR retinal image.

Clause 54. The retinal camera of clause 53, wherein the at least one property of the at least one IR or NIR retinal image comprises one or more of: retinal coverage, focus, illumination uniformity, glare detection, contrast, or retinal alignment and centering.

Clause 55. The retinal camera of any one of clauses 38 to 54, wherein the second quality assessment of the color retinal image comprises analyzing at least one property of the color retinal image.

Clause 56. The retinal camera of clause 55, wherein the at least one property of the color retinal image comprises one or more of: clarity and sharpness, color balance and contrast, retinal coverage, presence of artifacts, or diagnostic adequacy of disease identification.

Clause 57. A method of operating the retinal camera of any one of clauses 38 to 56.

Clause 58. A non-transitory computer readable medium storing instructions that, when executed by an electronic processing circuitry comprising, cause the electronic processing circuitry to execute the method of clause 57.

Clause 59. A retinal camera comprising: a housing; an infrared (IR) or near infrared (NIR) light source supported by the housing and configured to provide continuous, non-mydriatic illumination for capturing IR or NIR retinal images without pharmacological pupil dilation; a visible light source supported by the housing and configured to provide flash illumination for capturing color retinal images; at least one image sensor supported by the housing and configured to capture IR or NIR retinal images and color retinal images; a user interface supported by the housing; an on-board hardware accelerator supported by the housing and configured to implement: a first Holistic Quality Assessor (HQA-1) comprising at least one machine-learning model that is optimized for embedded inference and trained on a dataset that includes IR or NIR retinal images, the HQA-1 being configured to assess quality of the IR or NIR retinal images captured by the at least one image sensor by: 1) determining whether at least one property of a first IR or NIR retinal image satisfies a first quality threshold and 2) responsive to determining that the at least one property of the first IR or NIR retinal image satisfies the first quality threshold, provide an indication that a color retinal image should be captured; and a second Holistic Quality Assessor (HQA-2) comprising at least one machine-learning model that is optimized for embedded inference and trained on a dataset that includes color retinal images exhibiting multiple types of quality defects, the HQA-2 configured to: 1) perform a multi-factor or holistic quality assessment of the color retinal image by determining whether at least one property of the color retinal image satisfies a second quality threshold and 2) responsive to determining that the at least one property of the color retinal image satisfies the second quality threshold, provide an indication the color retinal image is of diagnostic quality; and a processor supported by the housing and configured to communicate with the on-board hardware accelerator and to control the IR or NIR light source, the visible light source, the at least one image sensor, and the user interface, the processor being further configured to: responsive to receiving the indication that the color retinal image should be captured, automatically cause activation of the visible light source and capture of the color retinal image by the at least one image sensor; and responsive to receiving the indication that the color retinal image is of diagnostic quality, cause a user interface to provide a notification that a color retinal image of diagnostic quality has been captured, wherein the HQA-1 and HQA-2 are optimized to perform all image quality assessments solely on the on-board hardware accelerator without transmitting any of the IR or NIR retinal images or any of the color retinal images to any external computing device for quality assessment thereby enabling low-latency local and sequential assessment of retinal image quality by the HQA-1 and HQA-2 and capture of diagnostic quality color retinal images with reduced or minimal manual intervention by an operator.

Clause 60. The retinal camera of clause 59, wherein the HQA-1 is configured to: assess quality of a second IR or NIR retinal image responsive to determining that the at least one property of the first IR or NIR retinal image fails to satisfy the first quality threshold, the second IR or NIR retinal image captured subsequent to capture of the first IR or NIR retinal image; and responsive to determining that at least one property of the second IR or NIR retinal image satisfies the first quality threshold, provide the indication that the color retinal image should be captured.

Clause 61. The retinal camera of any one of clauses 59 to 60, wherein the HQA-2 is configured to cause the HQA-1 to assess quality of a second IR or NIR retinal image responsive to determining that the at least one property of the color retinal image fails to satisfy the second quality threshold, the second IR or NIR retinal image captured subsequent to capture of the first IR or NIR retinal image.

Clause 62. The retinal camera of any one of clauses 59 to 61, wherein the processor is configured to include in the notification an indication to discard the color retinal image or retain the color retinal image after additional review responsive to receiving an indication from the HQA-2 that the at least one property of the color retinal image fails to satisfy the second quality threshold.

Clause 63. The retinal camera of any one of clauses 59 to 62, wherein the processor is configured to control the IR or NIR light source, the visible light source, and the at least one image sensor to: operate in a first mode in which the IR or NIR retinal images are continuously captured for quality assessment by the HQA-1; and operate in a second mode in which the color retinal image is automatically captured responsive to determining, by the HQA-1, that the at least one property of the first IR or NIR retinal image satisfies the first quality threshold.

Clause 64. The retinal camera of clause 63, wherein, in the second mode, capture of the IR or NIR retinal images is paused.

Clause 65. The retinal camera of any of clauses 59 to 64, wherein the HQA-1 comprises at least one first deep-learning model and the HQA-2 comprises at least one second deep-learning model, and wherein the at least one first or second deep-learning model has been pruned or quantized to reduce power consumption, processing latency, and memory storage.

Clause 66. The retinal camera of any one of clauses 59 to 65, wherein the HQA-1 is configured to operate in multiple modes comprising: quality assessment of each image frame of an IR or NIR retinal video stream in real time; quality assessment of every Nth image frame of the IR or NIR retinal video stream, wherein N is an integer greater than 1; quality assessment of image frames of the IR or NIR retinal video stream during a predetermined time window followed by a pause; and quality assessment of an IR or NIR retinal image responsive to a request from the operator.

Clause 67. The retinal camera of any one clauses 59 to 66, wherein the processor is further configured to implement a Real-Time Imaging Optimizer (RIO) to dynamically adjust one or more imaging parameters based on feedback from the HQA-1 to enhance image quality and improve the likelihood of capturing diagnostic quality color retinal images.

Clause 68. The retinal camera of clause 67, wherein the one or more imaging parameters comprise focus, IR or NIR light intensity, or exposure.

Clause 69. The retinal camera of clause 67 or 68, wherein the RIO is configured to: operate only in response to an instruction from the HQA-1; and perform adjustment of at least imaging parameter of the one or more imaging parameters identified by the HQA-1 as contributing to suboptimal retinal image quality to optimize imaging conditions for subsequent capture of IR or NIR retinal images to increase the likelihood of satisfying the first quality threshold.

Clause 70. The retinal camera of clause 69, wherein the HQA-1 is configured to determine one or more quality deficiencies responsive to the first IR or NIR retinal image failing to satisfy the first quality threshold and to activate the RIO based on the one or more quality deficiencies.

Clause 71. The retinal camera of any one clauses 59 to 70, wherein the processor is configured to: cause the user interface configured to display real-time feedback on alignment of the housing with respect to an eye thus enabling the operator to facilitate accurate and efficient image capture.

Clause 72. The retinal camera of any one clauses 59 to 71, wherein the at least one property of the first IR or NIR retinal image comprises one or more of focus, alignment, or glare, and wherein the at least one property of the color retinal image comprises one or more of clarity, contrast, retinal coverage, or presence of artifacts.

Clause 73. A method of operating a retinal camera, the method comprising: at a first time, by an on-board hardware accelerator: with a first Holistic Quality Assessor (HQA-1) comprising at least one machine-learning model that is optimized for embedded inference and trained on a dataset that includes IR or NIR retinal images, assessing quality of infrared (IR) or near infrared (NIR) retinal images captured as a result of illuminating a retina with continuous, non-mydriatic illumination, the assessing comprising: 1) determining whether at least one property of a first IR or NIR retinal image satisfies a first quality threshold and 2) responsive to determining that the at least one property of the first IR or NIR retinal image satisfies the first quality threshold, providing an indication that a color retinal image should be captured; at a second time, by an on-board processor: responsive to receiving the indication that the color retinal image should be captured, automatically causing illumination of the retina with flash illumination and capture of the color retinal image; and at a third time, by the on-board hardware accelerator: with a second Holistic Quality Assessor (HQA-2) comprising at least one machine-learning model that is optimized for embedded inference and trained on a dataset that includes color retinal images exhibiting multiple types of quality defects: 1) determining whether at least one property of the color retinal image satisfies a second quality threshold and 2) responsive to determining that the at least one property of the color retinal image satisfies the second quality threshold, providing an indication the color retinal image is of diagnostic quality and causing the on-board processor to provide a notification that a color retinal image of diagnostic quality has been captured, wherein the HQA-1 and HQA-2 are optimized to perform all image quality assessments solely on the on-board hardware accelerator without transmitting any of the IR or NIR retinal images or any of the color retinal images to any external computing device for quality assessment thereby enabling low-latency local and sequential assessment of retinal image quality by the HQA-1 and HQA-2 and capture of diagnostic quality color retinal images with reduced or minimal manual intervention by an operator.

Clause 74. The method of clause 73, further comprising: at a fourth time, by the on-board hardware accelerator: with the HQA-1, determining that the at least one property of the first IR or NIR retinal image fails to satisfy the first quality threshold; with the HQA-1, in response to determining that the at least one property of the first IR or NIR retinal image fails to satisfy the first quality threshold, assessing quality of a second IR or NIR retinal image captured subsequent to capture of the first IR or NIR retinal image; with the HQA-1, determining that at least one property of the second IR or NIR retinal image satisfies the first quality threshold; and with the HQA-1, in response to determining that at least one property of the second IR or NIR retinal image satisfies the first quality threshold, provide the indication that the color retinal image should be captured.

Clause 75. The method of any one of clauses 73 to 74, further comprising: at a fourth time, by the on-board hardware accelerator: with the HQA-2, determining that the at least one property of the color retinal image fails to satisfy the second quality threshold; and with the HQA-2, in response to determining that the at least one property of the color retinal image fails to satisfy the second quality threshold, causing the HQA-1 to assess quality of a second IR or NIR retinal image captured subsequent to capture of the first IR or NIR retinal image.

Clause 76. The method of any one of clauses 73 to 75, further comprising: at a fourth time, by the on-board processor: receiving an indication from the HQA-2 that the at least one property of the color retinal image fails to satisfy the second quality threshold; and in response to receiving the indication that the at least one property of the color retinal image fails to satisfy the second quality threshold, including in the notification an indication to discard the color retinal image or retain the color retinal image after additional review.

Clause 77. The method of any clauses 73 to 76, wherein the HQA-1 comprises at least one first deep-learning model and the HQA-2 comprises at least one second deep-learning model, and wherein the at least one first or second deep-learning model has been pruned or quantized to reduce power consumption, processing latency, and memory storage.

Clause 78. The method of any one clauses 73 to 77, wherein the HQA-1 operates in multiple modes comprising: quality assessment of each image frame of an IR or NIR retinal video stream in real time; quality assessment of every Nth image frame of the IR or NIR retinal video stream, wherein N is an integer greater than 1; quality assessment of image frames of the IR or NIR retinal video stream during a predetermined time window followed by a pause; and quality assessment of an IR or NIR retinal image responsive to a request from the operator.

Clause 79. The method of any clauses 73 to 78, further comprising: by the on-board processor: executing a Real-Time Imaging Optimizer (RIO) to dynamically adjust one or more imaging parameters based on feedback from the HQA-1 to enhance image quality and improve the likelihood of capturing diagnostic quality color retinal images.

Clause 80. The method of clause 79, wherein the one or more imaging parameters comprise focus, IR or NIR light intensity, or exposure.

Clause 81. The method of clause 79 or 80, further comprising: at a third time, by the on-board hardware accelerator: with the HQA-1, determining that the first IR or NIR retinal image fails to satisfy the first quality threshold; and with the HQA-1, in response to determining that the first IR or NIR retinal image fails to satisfy the first quality threshold, determining one or more quality deficiencies and activating the RIO based on the one or more quality deficiencies.

Clause 82. The method of any one clauses 73 to 81, wherein the at least one property of the first IR or NIR retinal image comprises one or more of focus, alignment, or glare, and wherein the at least one property of the color retinal image comprises one or more of clarity, contrast, retinal coverage, or presence of artifacts.

Clause 83. A non-transitory computer readable medium storing instructions that, when executed by an on-board processing circuitry of a retinal camera, cause the processing circuitry to implement the method of any one of clauses 73 to 82.

### Terminology

All of the methods and tasks described herein can be performed and fully automated by a computer-implemented system. The computer-implemented system may be local. In some cases, the computer system may include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid-state storage devices, disk drives, etc.). The various functions disclosed herein can be embodied in such program instructions or can be implemented in application-specific circuitry (e.g., ASICs or FPGAs) of the computer system. Where the computer system includes multiple computing devices, these devices may, but need not, be co-located. The results of the disclosed methods and tasks can be persistently stored by transforming physical storage devices, such as solid-state memory chips or magnetic disks, into a different state. In some cases, the computer system can be a cloud-based computing system whose processing resources are shared by multiple distinct business entities or other users.

Depending on the implementation, certain acts, events, or functions of any of the processes or algorithms described herein can be performed in a different sequence, can be added, merged, or left out altogether (e.g., not all described operations or events are necessary for the practice of the algorithm). Moreover, in certain cases, operations or events can be performed concurrently, e.g., through multi-threaded processing, interrupt processing, multiple processors or processor cores, or on other parallel architectures rather than sequentially

The various illustrative logical blocks, modules, routines, and algorithm steps described in connection with the examples disclosed herein can be implemented as electronic hardware (e.g., ASICs or FPGA devices), computer software that runs on computer hardware, or combinations of both. Moreover, the various illustrative logical blocks and modules described in connection with the examples disclosed herein can be implemented or performed by a machine, such as a processor device, a digital signal processor (DSP), an application-specific integrated circuit (ASIC), a field programmable gate array (FPGA) or other programmable logic devices, discrete gate or transistor logic, discrete hardware components, or any combination thereof designed to perform the functions described herein. For example, a processor device can be a microprocessor, but in the alternative, the processor device can be a controller, microcontroller, or logic circuitry that implements a state machine, combinations of the same, or the like. A processor device can include electrical circuitry configured to process computer-executable instructions. In another instance, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device can also include primarily analog components. For example, some or all of the rendering techniques described herein can be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the examples disclosed herein can be embodied directly in hardware, a firmware or software module executed by a processor device, or a combination of the two. A firmware or software module can reside in RAM memory, flash memory, ROM memory, EPROM memory, EEPROM memory, registers, hard disk, a removable disk, a CD-ROM, or any other form of a non-transitory computer-readable storage medium. An example storage medium can be coupled to the processor device such that the processor device can read information from, and write information to, the storage medium. Alternatively, the storage medium can be integral to the processor device. For example, the processor device and the storage medium can reside in an ASIC. The ASIC can reside in a user terminal. Alternatively, the processor device and the storage medium can reside as discrete components in a user terminal.

Conditional language used herein, such as, among others, “can,” “can,” “might,” “may,” “e.g.,” and the like, unless specifically stated otherwise or otherwise understood within the context as used, is generally intended to convey that certain implementations include, while other implementations do not include certain features, elements or steps. Thus, such conditional language is not generally intended to imply that features, elements, or steps are in any way required for one or more implementations or that one or more implementations necessarily include logic for deciding, with or without other input or prompting, whether these features, elements or steps are included or are to be performed in any particular example. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive languages such as the phrase “at least one of X, Y, or Z,” unless specifically stated otherwise, is otherwise understood with the context as used in general to present that an item, term, etc., can be either X, Y, or Z, or any combination thereof (e.g., X, Y, or Z). Thus, such disjunctive language is not generally intended to, and should not, imply that certain cases require at least one of X, at least one of Y, and at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C. Unless otherwise explicitly stated, the terms “set” and “collection” should generally be interpreted to include one or more described items throughout this application. Accordingly, phrases such as “a set of devices configured to” or “a collection of devices configured to” are intended to include one or more recited devices. Such one or more recited devices can also be collectively configured to carry out the stated recitations. For example, “a set of servers configured to carry out recitations A, B and C” can include a first server configured to carry out recitation A working in conjunction with a second server configured to carry out recitations B and C.

While the above-detailed description has shown, described, and pointed out novel features as applied to various examples, it can be understood that various omissions, substitutions, and changes in the form and details of the devices or algorithms illustrated can be made without departing from the spirit of the disclosure. As can be recognized, certain examples described herein can be embodied within a form that does not provide all of the features and benefits set forth herein, as some features can be used or practiced separately from others. Accordingly, the scope of certain examples disclosed herein is indicated by the appended claims rather than by the foregoing description. All changes which come within the meaning and range of equivalency of the claims are to be embraced within their scope.

What is claimed is:

1. A retinal camera comprising:
a housing;
at least one infrared (IR) or near infrared (NIR) light source supported by the housing and configured to provide continuous, non-mydriatic illumination for capturing IR or NIR retinal images of an eye;
at least one visible light source supported by the housing and configured to provide flash illumination for capturing color retinal images of the eye;
at least one image sensor supported by the housing and configured to capture the IR or NIR retinal images and color retinal images;
a user interface supported by the housing; and
on-board processing circuitry supported by the housing and comprising at least one processor and a hardware accelerator, the on-board processing circuitry configured to:
implement a first Holistic Quality Assessor (HQA-1) comprising at least one first machine learning model that is configured to be executed on the hardware accelerator, the at least one first machine learning model trained on a dataset that includes IR or NIR retinal images, the HQA-1 being configured to: 1) perform real-time quality assessments of the IR or NIR retinal images captured by the at least one image sensor by determining whether at least one property of the IR or NIR retinal images satisfies a first quality threshold and 2) generate quality assessments for a plurality of IR or NIR retinal images;
generate an indication that a color retinal image should be captured based on the first quality threshold being satisfied for at least two IR or NIR retinal images captured at different times within a time window;
responsive to the indication that the color retinal image should be captured, cause activation of the at least one visible light source to emit one or more flash illumination events during an acquisition interval and cause capture of one or more color retinal image frames by the at least one image sensor;
generate an output color retinal image by selecting one of the one or more color retinal image frames or by combining at least two of the one or more color retinal image frames;
implement a second Holistic Quality Assessor (HQA-2) comprising at least one second machine learning model that is configured to be executed on the hardware accelerator, the at least one second machine learning model trained on a dataset that includes color retinal images, the HQA-2 configured to: 1) determine whether at least one property of the output color retinal image satisfies a second quality threshold and 2) generate an indication that the output color retinal image is of diagnostic quality responsive to determining that the at least one property satisfies the second quality threshold; and
responsive to generating the indication that the output color retinal image is of diagnostic quality, cause the user interface to provide a notification that the color retinal image of diagnostic quality has been captured,
wherein the quality assessments performed by the HQA-1 and HQA-2 are performed locally by the on-board processing circuitry without transmitting any of the IR or NIR retinal images or any of the color retinal images to an external computing device for quality assessment.

2. The retinal camera of claim 1, wherein the on-board processing circuitry is further configured to implement a capture gate configured to determine that the first quality threshold has been satisfied for a threshold number of consecutive or non-consecutive IR or NIR retinal images within the time window prior to activation of the at least one visible light source.

3. The retinal camera of claim 1, wherein the at least one visible light source is configured to emit a plurality of time-multiplexed sub-flashes within a capture window while the at least one image sensor captures multiple distinct color retinal image frames, at least two color retinal image frames being acquired with different exposure parameters.

4. The retinal camera of claim 3, wherein plurality of time-multiplexed sub-flashes differ in at least one of: peak intensity, pulse width, spectral content, polarization state, or illumination geometry being adaptively selected based on at least one of: measured pupil size, a motion stability metric, ambient illumination, prior capture outcomes, or predicted glare risk.

5. The retinal camera of claim 1, wherein the on-board processing circuitry is further configured to:
cause capture of the one or more color retinal image frames further responsive to receiving a capture request being generated as a result of an operator manipulating the user interface;
responsive to receiving the capture request, latch the capture request so that the capture request remains asserted until the indication that the output color retinal image is of diagnostic quality has been generated; and
cancel the capture request responsive to a cancellation by the operator through the user interface or a timeout.

6. The retinal camera of claim 5, wherein the on-board processing circuitry is further configured to:
maintain the hardware accelerator in an inactive state so that power consumption is reduced;
transition the hardware accelerator to an active state and execute the HQA-1 responsive to receiving the capture request, wherein power consumption in the active state is greater than power consumption in the inactive state; and
transition the hardware accelerator to the inactive state responsive to completion of the quality assessment by the HQA-2 or a timeout.

7. The retinal camera of claim 1, wherein the on-board processing circuitry is further configured to:
responsive to the indication that the color retinal image should be captured, automatically cause activation of the at least one visible light source and capture of the one or more color retinal image frames.

8. The retinal camera of claim 1, wherein the on-board processing circuitry is further configured to:
responsive to the first quality threshold not being satisfied, adjust at least one imaging parameter of the at least one IR or NIR light source, the at least one imaging parameter comprising focus, IR or NIR light intensity, or exposure setting.

9. The retinal camera of claim 1, wherein the on-board processing circuitry is further configured to:
cause the user interface to output an indication to retake a color retinal image responsive to not generating the indication that the output color retinal image is of diagnostic quality, the indication to retake identifying at least one quality deficiency comprising alignment, illumination, non-uniformity, motion blur, glare, or insufficient retinal coverage.

10. The retinal camera of claim 1, wherein the on-board processing circuitry is further configured to:
determine a motion stability metric using at least one motion estimate determined from IR or NIR retinal images and measurements from at least motion sensor; and
cause activation of the at least one visible light source further responsive to the motion stability metric satisfying a stability threshold.

11. The retinal camera of claim 1, wherein the on-board processing circuitry is further configured to:
implement a feedback buffer that facilitates application of temporal smoothing to the quality assessments by the HQA-1 for providing feedback of IR or NIR retinal image capture on the user interface; and
implement an automated capture buffer that facilitates enforcing of multi-frame validation prior to causing activation of the at least one visible light source.

12. A non-transitory computer readable medium storing instructions that, when executed by a processing circuitry comprising at least one processor and a hardware accelerator, cause the processing circuitry to:
implement a first Holistic Quality Assessor (HQA-1) comprising at least one first machine learning model that is configured to be executed on the hardware accelerator, the at least one first machine learning model trained on a dataset that includes infrared (IR) or near infrared (NIR) retinal images, the HQA-1 being configured to: 1) perform real-time quality assessments of IR or NIR retinal images of an eye captured by at least one image sensor when at least one IR or NIR light source provides continuous, non-mydriatic illumination by determining whether at least one property of the IR or NIR retinal images of the eye satisfies a first quality threshold and 2) generate quality assessments for a plurality of IR or NIR retinal images of the eye;
generate an indication that a color retinal image should be captured based on the first quality threshold being satisfied for at least two IR or NIR retinal images of the eye captured at different times within a time window;
responsive to the indication that the color retinal image should be captured, cause activation of at least one visible light source to emit one or more flash illumination events during an acquisition interval and cause capture of one or more color retinal image frames by the at least one image sensor;
generate an output color retinal image by selecting one of the one or more color retinal image frames or by combining at least two of the one or more color retinal image frames;
implement a second Holistic Quality Assessor (HQA-2) comprising at least one second machine learning model that is configured to be executed on the hardware accelerator, the at least one second machine learning model trained on a dataset that includes color retinal images, the HQA-2 configured to: 1) determine whether at least one property of the output color retinal image satisfies a second quality threshold and 2) generate an indication that the output color retinal image is of diagnostic quality responsive to determining that the at least one property satisfies the second quality threshold; and
responsive to generating the indication that the output color retinal image is of diagnostic quality, to provide a notification that the color retinal image of diagnostic quality has been captured,
wherein the quality assessments performed by the HQA-1 and HQA-2 are performed locally by the processing circuitry without transmitting any of the IR or NIR retinal images or any of the color retinal images to an external computing device for quality assessment.

13. The computer readable medium of claim 12, wherein the processing circuitry is further caused to implement a capture gate configured to determine that the first quality threshold has been satisfied for a threshold number of consecutive or non-consecutive IR or NIR retinal images within the time window prior to activation of the at least one visible light source.

14. The computer readable medium of claim 12, wherein the at least one visible light source is configured to emit a plurality of time-multiplexed sub-flashes within a capture window while the at least one image sensor captures multiple distinct color retinal image frames, at least two color retinal image frames being acquired with different exposure parameters.

15. The computer readable medium of claim 12, wherein the processing circuitry is further caused to:
cause capture of the one or more color retinal image frames further responsive to receiving a capture request being generated as a result of an operator manipulating a user interface;
responsive to receiving the capture request, latch the capture request so that the capture request remains asserted until the indication that the output color retinal image is of diagnostic quality has been generated; and
cancel the capture request responsive to a cancellation by the operator through the user interface or a timeout.

16. The computer readable medium of claim 15, wherein the processing circuitry is further caused to:
maintain the hardware accelerator in an inactive state so that power consumption is reduced;
transition the hardware accelerator to an active state and execute the HQA-1 responsive to receiving the capture request, wherein power consumption in the active state is greater than power consumption in the inactive state; and
transition the hardware accelerator to the inactive state responsive to completion of the quality assessment by the HQA-2 or a timeout.

17. The computer readable medium of claim 12, wherein the processing circuitry is further caused to:
responsive to the indication that the color retinal image should be captured, automatically cause activation of the at least one visible light source and capture of the one or more color retinal image frames.

18. The computer readable medium of claim 12, wherein the processing circuitry is further caused to:
responsive to the first quality threshold not being satisfied, adjust at least one imaging parameter of the at least one IR or NIR light source, the at least one imaging parameter comprising focus, IR or NIR light intensity, or exposure setting.

19. The computer readable medium of claim 12, wherein the processing circuitry is further caused to:
cause a user interface to output an indication to retake a color retinal image responsive to not generating the indication that the output color retinal image is of diagnostic quality, the indication to retake identifying at least one quality deficiency comprising alignment, illumination, non-uniformity, motion blur, glare, or insufficient retinal coverage.

20. The computer readable medium of claim 12, wherein the processing circuitry is further caused to:
determine a motion stability metric using at least one motion estimate determined from IR or NIR retinal images and measurements from at least motion sensor; and
cause activation of the at least one visible light source further responsive to the motion stability metric satisfying a stability threshold.
